(19) United States
(12) Patent Application Publication
(10) Pub. No.: US 2009/0269796 A1
(43) Pub. Date: Oct. 29, 2009

(54) METHODS OF DETECTING AND TREATING MYOCARDIAL ISCHEMIA AND MYOCARDIAL INFARCTION

(75) Inventors: Robert Gerszten, Brookline, MA (US); Marc S. Sabatine, Newton, MA (US); Michael A. Fifer, Brookline, MA (US)

Correspondence Address: EWARDS ANGELL PALMER & DODGE LLP, P.O. BOX 55874, BOSTON, MA, 02205, US

(73) Assignee: THE GENERAL HOSPITAL CORPORATION, Boston, MA (US)

(21) Appl. No.: 12/277,740

(22) Filed: Nov. 25, 2008

Related U.S. Application Data
(60) Provisional application No. 60/992,526, filed on Dec. 5, 2007.

(51) Int. Cl. C12Q 1/02

(52) U.S. Cl. 435/29

(57) ABSTRACT

Methods of detecting and treating myocardial ischemia and myocardial infarction based on the differential expression of metabolic products are described herein.

### CROSS-REFERENCE TO RELATED APPLICATIONS/PATENTS & INCORPORATION BY REFERENCE

This application claims priority to U.S. provisional application Ser. No. 60/992,526, filed Dec. 5, 2007. This application also contains subject matter that is related to the subject matter disclosed and claimed in WO2006/036476, published on Apr. 6, 2006. The entire disclosures of the aforementioned patent applications are incorporated herein by this reference.

Each of the applications and patents cited in this text, as well as each document or reference cited in each of the applications and patents (including during the prosecution of each issued patent; “application cited documents”), and each of the PCT and foreign applications/publications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List before the paragraphs, or in the text itself; and, each of these documents or references (“herein-cited references”), as well as each document or reference cited in each of the herein-cited references (including any manufacturer's specifications, instructions, etc.), is hereby expressly incorporated herein by reference.

### STATEMENT OF RIGHTS TO INVENTIONS MADE UNDER FEDERALLY SPONSORED RESEARCH

This work was supported by National Institutes of Health Grant Nos. F32 HL68455 and R01HL072872 and R01 HL083141. The Government has certain rights in the invention.

### BACKGROUND OF THE INVENTION

Coronary artery disease is a leading cause of morbidity and mortality worldwide. Recognition of myocardial ischemia is critical both for diagnosing coronary heart disease and for selecting and evaluating the response to therapeutic interventions. Currently, myocardial ischemia is diagnosed through a combination of a history consistent with typical angina pectoris and labile electrocardiographic ST-segment and T wave changes, occurring either spontaneously or upon provocation with exercise testing (Gibbons, R., et al. 2002 ACC/ACA guideline update for the management of patients with chronic stable angina: a report of the American College of Cardiology/American Heart Association Task Force on Practice Guidelines; available upon request to ACC; Braunwald, E., et al. 2002 ACC/ACA guideline update for the management of patients with unstable angina and non-ST-segment elevation myocardial infarction: a report of the American College of Cardiology/American Heart Association Task Force on Practice Guidelines; available upon request to ACC). This approach, however, is often unsatisfactory due to the transient nature of electrocardiographic changes, as well as the subjective nature of history taking, particularly in the growing diabetic and elderly populations in whom symptoms are often atypical. Exercise testing with myocardial perfusion imaging is relatively accurate, but adds over $2000 to the cost and is difficult to implement rapidly in settings such as the emergency department (Gibbons, R., et al. 1997 J Am Coll Cardiol 30:260-311; Ritchie, J. L., et al. 1995 J Nucl Cardiol. 2:172-92). Although several biomarkers accurately diagnose patients with irreversible injury secondary to myocardial infarction, none is suitable for detecting the more subtle insult of myocardial ischemia (Morrow, D. A., et al. 2003 Clin Chem 49:537-9).

Acute Myocardial Infarction (MI) is the leading cause of death in the United States, with 500,000 of the approximately 1.1 million attacks each year being fatal (Braunwald, E., et al. 2000 J Am Coll Cardiol 36:970-1062). The steep time-to-treatment benefit curve underlying current reperfusion strategies exemplifies how early, reliable diagnosis of acute coronary syndromes has acquired not only prognostic, but also increasingly therapeutic importance (Fibrinolytic Therapy Trialists' (FTT) Collaborative Group 1994 Lancet 343:311-322; Morrison, L. J. et al. 2000 JAMA 283:2686-92; Bonnefoy, E., et al. 2002 Lancet 360:825-9; Cannon, C. P., et al. 2000 JAMA 283:2941-7; Neumann, F. J., et al. 2003 JAMA 290:1593-9). However, conventional evaluations based on symptoms, physical examination and electrocardiographic findings are often inconclusive, particularly in aging and diabetic populations with preexisting coronary artery disease. Furthermore, available serum markers of myocardial infarction such as the troponins have limited sensitivity and specificity in the first several hours following the onset of injury (Braunwald, E., et al. 2000 J Am Coll Cardiol 36:970-1062).

Recent advances in proteomic and metabolic profiling technologies have enhanced the feasibility of high throughput patient screening for the diagnosis of disease states (Nicholson, J. K., et al. 2003 Nat Rev Drug Discov 2:668-76). The profiling of low molecular-weight metabolic products is particularly relevant to exercise physiology and myocardial ischemia. Small biochemicals are the end result of the entire chain of regulatory changes that occur in response to physiological stressors, disease processes, or drug therapy. In addition to serving as biomarkers, circulating metabolic products may themselves participate as regulatory signals such as in the control of blood pressure (He, W., et al. 2004 Nature 429:188-93).

### SUMMARY OF THE INVENTION

Circulating metabolic products that change depending on the presence of myocardial ischemia and myocardial infarction have now been identified and characterized. Such products can serve as targets for therapeutic intervention or as substrates for molecular imaging.

In one aspect, the invention provides a method of detecting myocardial ischemia or myocardial infarction in a subject comprising detecting in a biological sample obtained from the subject a change in the amount of at least one member selected from the group consisting of malonic acid, asymmetric dimethyl arginine (ADMA), succinic acid, anthanilic acid, acetyl-CoA, asymmetric dimethyl arginine (ADMA)/symmetric dimethyl arginine (SDMA), and a metabolic product thereof, thereby detecting myocardial ischemia or early myocardial infarction in the subject. In additional embodiments of a method of the invention, the change detected is in the amount of malonic acid or a metabolic product thereof, or in the amount of asymmetric dimethyl arginine (ADMA) or a metabolic product thereof, or in the amount of succinic acid or a metabolic product thereof, or in the amount of anthanilic acid or a metabolic product thereof, or in the amount of acetyl-CoA or a metabolic product thereof, or in the amount of asymmetric dimethyl arginine (ADMA)/symmetric dimethyl arginine (SDMA) or a metabolic product thereof.

In yet another embodiment of a method of the invention, the change comprises a decrease in the amount of asymmetric dimethyl arginine (ADMA) or a metabolic product thereof. In still another embodiment of a method of the invention, the change comprises an increase in the amount of at least one member of the group consisting of malonic acid, succinic acid, anthanilic acid, acetyl-CoA, asymmetric dimethyl arginine (ADMA)/symmetric dimethyl arginine (SDMA), and a metabolic product thereof. In additional embodiments of a method of the invention, the change comprises an increase in the amount of malonic acid or a metabolic product thereof, or in the amount of succinic acid or a metabolic product thereof, or in the amount of anthinilic acid or a metabolic product thereof, or in the amount of acetyl-CoA or a metabolic product thereof, or in the amount of asymmetric dimethyl arginine (ADMA)/symmetric dimethyl arginine (SDMA) or a metabolic product thereof.

In a further embodiment of a method of the invention, the change comprises an increase in the amount of at least one member of the group consisting of malonic acid, succinic acid, anthinilic acid, acetyl-CoA, asymmetric dimethyl arginine (ADMA)/symmetric dimethyl arginine (SDMA), and a metabolic product thereof and a decrease in the amount of at least one of asymmetric dimethyl arginine (ADMA) or a metabolic product thereof.

In one embodiment of a method of the invention, myocardial infarction is detected. The myocardial infarction may be early myocardial infarction. In another embodiment of a method of the invention, myocardial ischemia is detected.

In one embodiment of a method of the invention, the biological sample comprises a blood sample or a preparation thereof. The preparation may comprise plasma or serum.

In one embodiment of a method of the invention, the subject is a human.

In a further embodiment of a method of the invention, the change is detected after administration of a controlled ischemic insult or planned myocardial infarction to the subject. The controlled ischemic insult may comprise exercise testing. The planned myocardial infarction may comprise alcohol septal ablation for hypertrophic cardiomyopathy.

In still a further embodiment of a method of the invention, the detecting comprises analyzing the sample, or a preparation thereof, using liquid chromatography and mass spectrometry. The mass spectrometry may comprise high sensitivity electrospray mass spectrometry.

In another aspect, the invention provides a metabolic profile indicating myocardial ischemia or myocardial infarction in a subject comprising a change in the amount of at least one member of the group consisting of malonic acid, asymmetric dimethyl arginine (ADMA), succinic acid, anthanilic acid, acetyl-CoA, asymmetric dimethyl arginine (ADMA)/symmetric dimethyl arginine (SDMA), and a metabolic product thereof in a biological sample obtained from the subject. In additional embodiments of a profile of the invention, the change is in the amount of malonic acid or a metabolic product thereof, or in the amount of asymmetric dimethyl arginine (ADMA) or a metabolic product thereof, or in the amount of succinic acid or a metabolic product thereof, or in the amount of anthanilic acid or a metabolic product thereof, or in the amount of acetyl-CoA or a metabolic product thereof, or in the amount of asymmetric dimethyl arginine (ADMA)/symmetric dimethyl arginine (SDMA) or a metabolic product thereof.

In yet another embodiment of a profile of the invention, the change comprises a decrease in the amount of asymmetric dimethyl arginine (ADMA) or a metabolic product thereof. In still another embodiment of a profile of the invention, the change comprises an increase in the amount of at least one member of the group consisting of malonic acid, succinic acid, anthanilic acid, acetyl-CoA, asymmetric dimethyl arginine (ADMA)/symmetric dimethyl arginine (SDMA), and a metabolic product thereof. In additional embodiments of a profiles of the invention, the change comprises an increase in the amount of malonic acid or a metabolic product thereof, or in the amount of succinic acid or a metabolic product thereof, or in the amount of anthanilic acid or a metabolic product thereof, or an increase in the amount of acetyl-CoA or a metabolic product thereof, or in the amount of asymmetric dimethyl arginine (ADMA)/symmetric dimethyl arginine (SDMA) or a metabolic product thereof.

In a further embodiment of a profile of the invention, the change comprises an increase in the amount of at least one member of the group consisting of malonic acid, succinic acid, anthanilic acid, acetyl-CoA, asymmetric dimethyl arginine (ADMA)/symmetric dimethyl arginine (SDMA), and a metabolic product thereof and a decrease in the amount of at least one of asymmetric dimethyl arginine (ADMA) or a metabolic product thereof.

In one embodiment of a profile of the invention, myocardial infarction is indicated. The myocardial infarction may be early myocardial infarction. In another embodiment of a profile of the invention, myocardial ischemia is indicated.

In one embodiment of a profile of the invention, the biological sample comprises a blood sample or a preparation thereof. The preparation may comprise plasma or serum.

In one embodiment of a profile of the invention, the subject is a human.

In a further embodiment of a profile of the invention, the change results from administration of a controlled ischemic insult or planned myocardial infarction to the subject. The controlled ischemic insult may comprise exercise testing. The planned myocardial infarction may comprise alcohol septal ablation for hypertrophic cardiomyopathy.

In yet another aspect, the invention provides a method of obtaining a metabolic profile of a subject afflicted with, or at risk of becoming afflicted with, myocardial ischemia or myocardial infarction, comprising the steps of:
i) analyzing a biological sample obtained from the subject; and
ii) detecting a change in the amount of at least one member of the group consisting of malonic acid, asymmetric dimethyl arginine (ADMA), succinic acid, anthanilic acid, acetyl-CoA, asymmetric dimethyl arginine (ADMA)/symmetric dimethyl arginine (SDMA), and a metabolic product thereof,
   thereby obtaining a metabolic profile of a subject afflicted with, or at risk of becoming afflicted with, myocardial ischemia or myocardial infarction. The myocardial infarction may be early myocardial infarction.

In one embodiment of a method of the invention, the biological sample is obtained from the subject before and after subjecting the subject to controlled ischemic insult or planned myocardial infarction. The controlled ischemic insult may comprise exercise testing. The planned myocardial infarction may comprise alcohol septal ablation for hypertrophic cardiomyopathy.

In another embodiment of a method of the invention, the analyzing comprises subjecting the sample, or a preparation thereof, to liquid chromatography and mass spectrometry. The mass spectrometry may comprise high sensitivity electrospray mass spectrometry.

In yet another aspect, the invention provides a method of treating myocardial ischemia or myocardial infarction in a subject, the method comprising administering to the subject a composition comprising a therapeutically effective amount of at least one compound selected from the group consisting of cholic acid, tyrosine, asymmetric dimethyl arginine (ADMA), sucrose, trimethylamine-N-oxide, orotic acid, malonic acid, inosine, glycerol-3-P, homoserine, threonine, choline, proline, creatine, 3-phosphoglyceric acid, ribose-5-P/ribulose-5-P, gamma-aminobutyric acid (GABA), oxaloacetate, citrulline, argininosuccinate, uric acid, citric acid, tryptophan, serine, uridine, and a metabolic product thereof, thereby treating myocardial ischemia or myocardial infarction in the subject.

In one embodiment of a treatment method of the invention, the at least one compound is selected from the group consisting of cholic acid, tyrosine, sucrose, trimethylamine-N-oxide, homoserine, threonine, choline, proline, creatine, 3-phosphoglyceric acid, ribose-5-P/ribulose-5-P, gamma-aminobutyric acid (GABA), oxaloacetate, citrulline, argininosuccinate, uric acid, citric acid, tryptophan, serine, uridine, and a metabolic product thereof. In another embodiment of a treatment method of the invention, the at least one compound is asymmetric dimethyl arginine (ADMA) or a metabolic product thereof. In another embodiment of a treatment method of the invention, the at least one compound is selected from the group consisting of cholic acid, tyrosine, asymmetric dimethyl arginine (ADMA), sucrose, trimethylamine-N-oxide, homoserine, threonine, choline, proline, creatine, 3-phosphoglyceric acid, ribose-5-P/ribulose-5-P, and a metabolic product thereof. In another embodiment of a treatment method of the invention, the at least one compound is selected from the group consisting of gamma-aminobutyric acid (GABA), oxaloacetate, citrulline, argininosuccinate, uric acid, citric acid, tryptophan, serine, uridine, and a metabolic product thereof. In another embodiment of a treatment method of the invention, the at least one compound is selected from the group consisting of trimethylamine-N-oxide, orotic acid, malonic acid, inosine, and glycerol-3-P.

In one embodiment, a treatment method of the invention further comprises obtaining the compound.

In yet another aspect, the invention provides a method of treating myocardial ischemia or myocardial infarction in a subject, the method comprising administering to the subject a composition comprising a therapeutically effective amount of an inhibitor of at least one compound selected from the group consisting of malonic acid, Ile/Leu, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, succinic acid, anthanilic acid, glycerol-3-P, glycerate-2-P, adenosine monophosphate (AMP), acetyl-CoA, asymmetric dimethyl arginine (ADMA)/symmetric dimethyl arginine (SDMA), malic acid, ascorbic acid, deoxycytidine monophosphate (DCMP), deoxycytidine diphosphate (DCDP), metanephrine, dimethyl glycine, lactic acid, hypoxanthine, taurine, inosine, alanine, phenylalanine, hydroxyhippuric acid, aconitic acid, and a metabolic product thereof, thereby myocardial ischemia or myocardial infarction in the subject.

In one embodiment of a treatment method of the invention, the at least one compound is selected from the group consisting of Ile/Leu, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, glycerol-3-P, glycerate-2-P, adenosine monophosphate (AMP), malic acid, ascorbic acid, deoxycytidine monophosphate (DCMP), deoxycytidine diphosphate (DCDP), metanephrine, dimethyl glycine, lactic acid, hypoxanthine, taurine, inosine, alanine, phenylalanine, hydroxyhippuric acid, aconitic acid, and a metabolic product thereof. In another embodiment of a treatment method of the invention, the at least one compound is selected from the group consisting of malonic acid, succinic acid, anthanilic acid, acetyl-CoA, asymmetric dimethyl arginine (ADMA)/symmetric dimethyl arginine (SDMA), and a metabolic product thereof. In another embodiment of a treatment method of the invention, the at least one compound is selected from the group consisting of malonic acid, Ile/Leu, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, succinic acid, glycerol-3-P, glycerate-2-P, adenosine monophosphate (AMP), acetyl-CoA, asymmetric dimethyl arginine (ADMA)/symmetric dimethyl arginine (SDMA), malic acid, ascorbic acid, deoxycytidine monophosphate (DCMP), deoxycytidine diphosphate (DCDP), metanephrine, dimethyl glycine, taurine, and a metabolic product thereof. In another embodiment of a treatment method of the invention, the at least one compound is selected from the group consisting of lactic acid, hypoxanthine, inosine, alanine, phenylalanine, hydroxyhippuric acid, aconitic acid, and a metabolic product thereof. In another embodiment of a treatment method of the invention, the at least one compound is selected from the group consisting of hypoxanthine, taurine, and succinic acid.

In one embodiment, a treatment method of the invention further comprises obtaining the inhibitor.

In yet another aspect, the invention provides a method of treating myocardial ischemia or myocardial infarction in a subject, the method comprising administering to the subject a composition comprising a therapeutically effective amount of: (i) at least one compound selected from the group consisting of cholic acid, tyrosine, asymmetric dimethyl arginine (ADMA), sucrose, trimethylamine-N-oxide, homoserine, threonine, choline, proline, creatine, 3-phosphoglyceric acid, ribose-5-P/ribulose-5-P, gamma-aminobutyric acid (GABA), oxaloacetate, citrulline, argininosuccinate, uric acid, citric acid, tryptophan, serine, uridine, and a metabolic product thereof; and (ii) an inhibitor of at least one compound selected from the group consisting of malonic acid, Ile/Leu, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, succinic acid, anthanilic acid, glycerol-3-P, glycerate-2-P, adenosine monophosphate (AMP), acetyl-CoA, asymmetric dimethyl arginine (ADMA)/symmetric dimethyl arginine (SDMA), malic acid, ascorbic acid, deoxycytidine monophosphate (DCMP), deoxycytidine diphosphate (DCDP), metanephrine, dimethyl glycine, lactic acid, hypoxanthine, taurine, inosine, alanine, phenylalanine, hydroxyhippuric acid, aconitic acid, and a metabolic product thereof, thereby treating myocardial ischemia or myocardial infarction in the subject.

In one embodiment, a treatment method of the invention further comprises obtaining the inhibitor and/or the compound(s). In another embodiment of a treatment method of the invention, the subject is a human. In yet another embodiment of a treatment method of the invention, the composition is administered to heart cells.

In still another aspect, the invention provides a method of preventing myocardial ischemia or myocardial infarction in a subject at risk for myocardial ischemia or myocardial infarction, the method comprising administering to the subject a composition comprising a therapeutically effective amount of at least one compound selected from the group consisting of cholic acid, tyrosine, asymmetric dimethyl arginine (ADMA), sucrose, trimethylamine-N-oxide, orotic acid, malonic acid, inosine, glycerol-3-P, homoserine, threonine, choline, proline, creatine, 3-phosphoglyceric acid, ribose-5-P/ribulose-5-P, gamma-aminobutyric acid (GABA), oxaloacetate, citrulline, argininosuccinate, uric acid, citric acid, tryptophan, serine, uridine, and a metabolic product thereof, thereby preventing myocardial ischemia or myocardial infarction in the subject at risk for myocardial ischemia or myocardial infarction.

In one embodiment of a treatment method of the invention, the at least one compound is selected from the group consisting of cholic acid, tyrosine, sucrose, trimethylamine-N-oxide, homoserine, threonine, choline, proline, creatine, 3-phosphoglyceric acid, ribose-5-P/ribulose-5-P, gamma-aminobutyric acid (GABA), oxaloacetate, citrulline, argininosuccinate, uric acid, citric acid, tryptophan, serine, uridine, and a metabolic product thereof. In another embodiment of a treatment method of the invention, the at least one compound is asymmetric dimethyl arginine (ADMA) or a metabolic product thereof. In another embodiment of a treatment method of the invention, the at least one compound is selected from the group consisting of cholic acid, tyrosine, asymmetric dimethyl arginine (ADMA), sucrose, trimethylamine-N-oxide, homoserine, threonine, choline, proline, creatine, 3-phosphoglyceric acid, ribose-5-P/ribulose-5-P, and a metabolic product thereof. In another embodiment of a treatment method of the invention, the at least one compound is selected from the group consisting of gamma-aminobutyric acid (GABA), oxaloacetate, citrulline, argininosuccinate, uric acid, citric acid, tryptophan, serine, uridine, and a metabolic product thereof. In another embodiment of a treatment method of the invention, the at least one compound is selected from the group consisting of trimethylamine-N-oxide, orotic acid, malonic acid, inosine, and glycerol-3-P.

In yet another aspect, the invention provides a method of preventing myocardial ischemia or myocardial infarction in a subject at risk for myocardial ischemia or myocardial infarction, the method comprising administering to the subject a composition comprising a therapeutically effective amount of an inhibitor of at least one compound selected from the group consisting of malonic acid, Ile/Leu, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, succinic acid, anthanilic acid, glycerol-3-P, glycerate-2-P, adenosine monophosphate (AMP), acetyl-CoA, asymmetric dimethyl arginine (ADMA)/symmetric dimethyl arginine (SDMA), malic acid, ascorbic acid, deoxycytidine monophosphate (DCMP), deoxycytidine diphosphate (DCDP), metanephrine, dimethyl glycine, lactic acid, hypoxanthine, taurine, inosine, alanine, phenylalanine, hydroxyhippuric acid, aconitic acid, and a metabolic product thereof, thereby preventing myocardial ischemia or myocardial infarction in the subject at risk for myocardial ischemia or myocardial infarction.

In one embodiment of a treatment method of the invention, the at least one compound is selected from the group consisting of Ile/Leu, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, glycerol-3-P, glycerate-2-P, adenosine monophosphate (AMP), malic acid, ascorbic acid, deoxycytidine monophosphate (DCMP), deoxycytidine diphosphate (DCDP), metanephrine, dimethyl glycine, lactic acid, hypoxanthine, taurine, inosine, alanine, phenylalanine, hydroxyhippuric acid, aconitic acid, and a metabolic product thereof. In another embodiment of a treatment method of the invention, the at least one compound is selected from the group consisting of malonic acid, succinic acid, anthanilic acid, acetyl-CoA, asymmetric dimethyl arginine (ADMA)/symmetric dimethyl arginine (SDMA), and a metabolic product thereof. In another embodiment of a treatment method of the invention, the at least one compound is selected from the group consisting of malonic acid, Ile/Leu, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, succinic acid, anthanilic acid, glycerol-3-P, glycerate-2-P, adenosine monophosphate (AMP), acetyl-CoA, asymmetric dimethyl arginine (ADMA)/symmetric dimethyl arginine (SDMA), malic acid, ascorbic acid, deoxycytidine monophosphate (DCMP), deoxycytidine diphosphate (DCDP), metanephrine, dimethyl glycine, taurine, and a metabolic product thereof. In another embodiment of a treatment method of the invention, the at least one compound is selected from the group consisting of lactic acid, hypoxanthine, inosine, alanine, phenylalanine, hydroxyhippuric acid, aconitic acid, and a metabolic product thereof. In another embodiment of a treatment method of the invention, the at least one compound is selected from the group consisting of hypoxanthine, taurine, and succinic acid.

In still another aspect, the invention provides a method of preventing myocardial ischemia or myocardial infarction in a subject at risk for myocardial ischemia or myocardial infarction, the method comprising administering to the subject a composition comprising (i) a therapeutically effective amount of at least one member of the group consisting of cholic acid, tyrosine, asymmetric dimethyl arginine (ADMA), sucrose, trimethylamine-N-oxide, homoserine, threonine, choline, proline, creatine, 3-phosphoglyceric acid, ribose-5-P/ribulose-5-P, gamma-aminobutyric acid (GABA), oxaloacetate, citrulline, argininosuccinate, uric acid, citric acid, tryptophan, serine, uridine, and a metabolic product thereof and (ii) a therapeutically effective amount of an inhibitor of at least one member of the group consisting of malonic acid, Ile/Leu, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, succinic acid, anthanilic acid, glycerol-3-P, glycerate-2-P, adenosine monophosphate (AMP), acetyl-CoA, asymmetric dimethyl arginine (ADMA)/symmetric dimethyl arginine (SDMA), malic acid, ascorbic acid, deoxycytidine monophosphate (DCMP), deoxycytidine diphosphate (DCDP), metanephrine, dimethyl glycine, lactic acid, hypoxanthine, taurine, inosine, alanine, phenylalanine, hydroxyhippuric acid, aconitic acid, and a metabolic product thereof, thereby preventing myocardial ischemia or myocardial infarction in the subject at risk for myocardial ischemia or myocardial infarction.

In yet another aspect, the invention provides a kit comprising (i) a therapeutically effective amount of at least one member of the group consisting of cholic acid, tyrosine, asymmetric dimethyl arginine (ADMA), sucrose, trimethylamine-N-oxide, homoserine, threonine, choline, proline, creatine, 3-phosphoglyceric acid, ribose-5-P/ribulose-5-P, gamma-aminobutyric acid (GABA), oxaloacetate, citrulline, argininosuccinate, uric acid, citric acid, tryptophan, serine, uridine, and a metabolic product thereof, (ii) a therapeutically effective amount of an inhibitor of at least one member of the group consisting of malonic acid, Ile/Leu, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, succinic acid, anthanilic acid, glycerol-3-P, glycerate-2-P, adenosine monophosphate (AMP), acetyl-CoA, asymmetric dimethyl arginine (ADMA)/symmetric dimethyl arginine (SDMA), malic acid, ascorbic acid, deoxycytidine monophosphate (DCMP), deoxycytidine diphosphate (DCDP), metanephrine, dimethyl glycine, lactic acid, hypoxanthine, taurine, inosine, alanine, phenylalanine, hydroxyhippuric acid, aconitic acid, and a metabolic product thereof, or (iii) a combination thereof and instructions for treating myocardial ischemia or myocardial infarction in a subject in accordance with the treatment methods described herein.

In still another aspect, the invention provides a kit comprising (i) a therapeutically effective amount of at least one member of the group consisting of cholic acid, tyrosine, asymmetric dimethyl arginine (ADMA), sucrose, trimethylamine-N-oxide, homoserine, threonine, choline, proline, taurine, creatine, 3-phosphoglyceric acid, ribose-5-P/ribulose-5-P, gamma-aminobutyric acid (GABA), oxaloacetate, citrulline, argininosuccinate, uric acid, citric acid, tryptophan, serine, uridine, and a metabolic product thereof, (ii) a therapeutically effective amount of an inhibitor of at least one member of the group consisting of malonic acid, Ile/Leu, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, succinic acid, anthanilic acid, glycerol-3-P, glycerate-2-P, adenosine monophosphate (AMP), acetyl-CoA, asymmetric dimethyl arginine (ADMA)/symmetric dimethyl arginine (SDMA), malic acid, ascorbic acid, deoxycytidine monophosphate (DCMP), deoxycytidine diphosphate (DCDP), metanephrine, dimethyl glycine, lactic acid, hypoxanthine, inosine, alanine, phenylalanine, hydroxyhippuric acid, aconitic acid, and a metabolic product thereof, or (iii) a combination thereof and instructions for preventing myocardial ischemia or myocardial infarction in a subject at risk for myocardial ischemia or myocardial infarction in accordance with the prevention methods described herein.

In still another aspect, the invention provides a method of detecting myocardial ischemia or myocardial infarction in a subject comprising detecting a decrease in the amount of at least one member of the group consisting of cholic acid, tyrosine, sucrose, trimethylamine-N-oxide, homoserine, threonine, choline, proline, 3-phosphoglyceric acid, and a metabolic product thereof in a biological sample obtained from the subject, thereby detecting myocardial ischemia or early myocardial infarction in the subject.

In still another aspect, the invention provides a method of detecting myocardial ischemia or myocardial infarction in a subject comprising detecting an increase in the amount of at least one member of the group consisting of Ile/Leu, taurine, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, glycerol-3-P, glycerate-2-P, adenosine monophosphate (AMP), malic acid, ascorbic acid, deoxycytidine monophosphate (DCMP), deoxycytidine diphosphate (DCDP), metanephrine, dimethyl glycine, creatine, ribose-5-P/ribulose-5-P, and a metabolic product thereof in a biological sample obtained from the subject, thereby detecting myocardial ischemia or early myocardial infarction in the subject.

In still another aspect, the invention provides a method of detecting myocardial ischemia or myocardial infarction in a subject comprising detecting a decrease in the amount of at least one member of the group consisting of cholic acid, tyrosine, sucrose, trimethylamine-N-oxide, homoserine, threonine, choline, proline, 3-phosphoglyceric acid, and a metabolic product thereof and an increase in the amount of at least one member of the group consisting of Ile/Leu, taurine, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, glycerol-3-P, glycerate-2-P, adenosine monophosphate (AMP), malic acid, ascorbic acid, deoxycytidine monophosphate (DCMP), deoxycytidine diphosphate (DCDP), metanephrine, dimethyl glycine, creatine, ribose-5-P/ribulose-5-P, and a metabolic product thereof in a biological sample obtained from the subject, thereby detecting myocardial ischemia or early myocardial infarction in the subject.

In still another aspect, the invention provides a metabolic profile indicating myocardial ischemia or myocardial infarction in a subject comprising a decrease in the amount of at least one member of the group consisting of cholic acid, tyrosine, sucrose, trimethylamine-N-oxide, homoserine, threonine, choline, proline, 3-phosphoglyceric acid, and a metabolic product thereof in a biological sample obtained from the subject.

In still another aspect, the invention provides a metabolic profile indicating myocardial ischemia or myocardial infarction in a subject comprising an increase in the amount of at least one member of the group consisting of Ile/Leu, taurine, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, glycerol-3-P, glycerate-2-P, adenosine monophosphate (AMP), malic acid, ascorbic acid, deoxycytidine monophosphate (DCMP), deoxycytidine diphosphate (DCDP), metanephrine, dimethyl glycine, creatine, ribose-5-P/ribulose-5-P, and a metabolic product thereof in a biological sample obtained from the subject.

In still another aspect, the invention provides a metabolic profile indicating myocardial ischemia or myocardial infarction in a subject comprising a decrease in the amount of at least one member of the group consisting of cholic acid, tyrosine, sucrose, trimethylamine-N-oxide, homoserine, threonine, choline, proline, 3-phosphoglyceric acid, and a metabolic product thereof and an increase in the amount of at least one member of the group consisting of Ile/Leu, taurine, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, glycerol-3-P, glycerate-2-P, adenosine monophosphate (AMP), malic acid, ascorbic acid, deoxycytidine monophosphate (DCMP), deoxycytidine diphosphate (DCDP), metanephrine, dimethyl glycine, creatine, ribose-5-P/ribulose-5-P, and a metabolic product thereof in a biological sample obtained from the subject.

Other aspects of the invention are described in or are obvious from the following disclosure, and are within the ambit of the invention.

### BRIEF DESCRIPTION OF THE FIGURES

The following Detailed Description, given by way of example, but not intended to limit the invention to specific embodiments described, may be understood in conjunction with the accompanying drawings, in which:

FIG. 1 schematically depicts the coronary sinus and femoral vein sampling.

FIG. 2 graphically depicts the circulating plasma levels of taurine and glutamic acid and confirms mass specrometer linear function in standard addition experiments.

FIG. 3 depicts, in bar graph form, the kinetics of metabolic changes in peripheral human plasma after planned myocardial injury. Representative metabolites demonstrating transient (≦120 minutes post-injury, Table 3), sustained (≧240 minutes), and late (1 day, 1440 minutes) changes in plasma levels compared to baseline are shown. Median±IQR (interquartile range, mean indicated by “+”) are shown for the entire cohort of 36 patients. Trimethylamine N-oxide is denoted as TMNO.

FIG. 4 depicts, in bar graph form, kinetic analyses of representative metabolites that are enriched in the coronary sinus after myocardial injury. As in Table 4, all metabolites listed show statistically significant changes in the coronary sinus at either the 10-minute or 60-minute time point, as compared to baseline. Black bars represent changes in coronary sinus levels; white bars represent changes in peripheral levels. * denotes significant differences in peripheral blood compared to baseline values (P<0.05). ^{#} denotes significant differences between coronary sinus and peripheral samples (P<0.05).

FIG. 5 graphically depicts, for metabolites that were significantly changed from baseline at 60, 120, and 240 minutes in planned MI patients and assessed in an independent spontaneous MI cohort, the averages of median changes across the three time points, as compared to baseline values in planned MIs (black bars) (upper panel). White bars denote relative levels of each of these metabolites in patients presenting with spontaneous MI, as compared to control patients presenting to the cardiac catheterization suite with non-acute cardiovascular disease. The lower left-hand panel depicts composite scores for the metabolites shown in the upper panel, derived by taking the weighted sum of metabolites that increase in planned MI minus the sum of metabolites that decrease in planned MI. The lower right-hand panel demonstrates the ROC curve for the composite score.

FIG. 6 shows, in bar-graph form, the mean % change in apoptosis, as compared to the hypoxia control, of neonatal rat cardiomyocytes pretreated with various individual metabolites.

### DETAILED DESCRIPTION OF THE INVENTION

### I. Definitions

Unless defined otherwise, all technical and scientific terms used herein have the meaning commonly understood by a person skilled in the art to which this invention belongs. The following references provide one of skill with a general definition of many of the terms used in this invention: Lackie and Dow, The Dictionary of Cell & Molecular Biology (3^{rd} ed. 1999); Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, “myocardial ischemia” refers to a disorder of cardiac function caused by insufficient blood flow to the muscle tissue of the heart. The decreased blood flow may, for example, be due to narrowing of the coronary arteries (coronary arteriosclerosis), to obstruction by a thrombus (coronary thrombosis), or less commonly, to diffuse narrowing of arterioles and other small vessels within the heart. Severe interruption of the blood supply to the myocardial tissue may result in necrosis of cardiac muscle (myocardial infarction).

As used herein, “myocardial infarction (MI)” refers to the irreversible necrosis of heart muscle secondary to prolonged ischemia. This usually results from an imbalance of oxygen supply and demand. The appearance of cardiac enzymes in the circulation generally indicates myocardial necrosis.

As used herein, “metabolite” refers to any substance produced or used during all the physical and chemical processes within the body that create and use energy, such as: digesting food and nutrients, eliminating waste through urine and feces, breathing, circulating blood, and regulating temperature. The term “metabolic precursors” refers to compounds from which the metabolites are made. The term “metabolic products” refers to any substance that is part of a metabolic pathway (e.g., metabolite, metabolic precursor).

As used herein, “biological sample” refers to a sample obtained from a subject. The biological sample can be selected, without limitation, from the group consisting of blood, plasma, serum, sweat, saliva, including sputum, urine, and the like. As used herein, “serum” refers to the fluid portion of the blood obtained after removal of the fibrin clot and blood cells, distinguished from the plasma in circulating blood. As used herein, “plasma” refers to the fluid, noncellular portion of the blood, distinguished from the serum obtained after coagulation.

As used herein, “subject” refers to any warm-blooded animal, particularly including a member of the class Mammalia such as, without limitation, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex and, thus, includes adult and newborn subjects, whether male or female.

As used herein, “treating” or “treatment” refers to ameliorating a condition, symptom, or parameter associated with an adverse heart condition such as myocardial ischemia or myocardial infarction or to preventing progression of myocardial ischemia or myocardial infarction, to either a statistically significant degree or to a degree detectable to one skilled in the art. By preventing progression of myocardial ischemia or myocardial infarction, a treatment can prevent deterioration of myocardial ischemia or myocardial infarction in an afflicted or diagnosed subject or a subject suspected of having myocardial ischemia or myocardial infarction, but, also, a treatment may prevent the onset of myocardial ischemia or myocardial infarction or a symptom thereof in a subject at risk for myocardial ischemia or myocardial infarction or suspected of having the same.

As used herein, “detecting” refers to methods that include identifying the presence or absence of substance(s) in the sample, quantifying the amount of substance(s) in the sample, and/or qualifying the type of substance. “Detecting” likewise refers to methods which include identifying the presence or absence of myocardial ischemia or early myocardial infraction in a subject, e.g., based upon the absence or presence of substances, e.g., metabolites and/or metabolic byproducts, in a sample obtained from a subject.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

“Obtaining” as in obtaining a compound refers to purchasing, synthesizing or otherwise acquiring the compound.

The terms “comprises”, “comprising”, and the like are intended to have the broad meaning ascribed to them in U.S. Patent Law and can mean “includes”, “including” and the like.

It is to be understood that this invention is not limited to the particular component parts of a device described or process steps of the methods described, as such devices and methods may vary. It is also to be understood that the terminology used herein is for purposes of describing particular embodiments only, and is not intended to be limiting. As used in the specification and the appended claims, the singular forms “a”, “an”, and “the” include plural referents unless the context clearly indicates otherwise.

### II. Embodiments of the Invention

### Sample Collection and Preparation

In one embodiment of the invention, the subject may undergo exercise testing after initial sample collection and before subsequent sample collection. In another embodiment of the invention, the subject may undergo a planned heart attack after initial sample collection and before subsequent sample collection.

In one embodiment of the invention, samples may be collected from individuals over a longitudinal period of time. Obtaining numerous samples from an individual over a period of time can be used to verify results from earlier detections and/or to identify an alteration in metabolite or polypeptide pattern as a result of, for example, pathology.

In one embodiment of the invention, the samples are analyzed without additional preparation and/or separation procedures.

In another embodiment of the invention, sample preparation and/or separation can involve, without limitation, any of the following procedures, depending on the type of sample collected and/or types of metabolic products searched: removal of high abundance polypeptides (e.g., albumin, and transferring; addition of preservatives and calibrants, desalting of samples; concentration of sample substances; protein digestions; and fraction collection. In yet another embodiment of the invention, sample preparation techniques concentrate information-rich metabolic products and deplete metabolites or other substances that would carry little or no information such as those that are highly abundant or native to serum.

In another embodiment of the invention, sample preparation takes place in a manifold or preparation/separation device. Such a preparation/separation device may, for example, be a microfluidics device. In yet another embodiment of the invention, the preparation/separation device interfaces directly or indirectly with a detection device. Such a preparation/separation device may, for example, be a fluidics device.

In another embodiment of the invention, the removal of undesired polypeptides (e.g., high abundance, uninformative, or undetectable polypeptides) can be achieved using high affinity reagents, high molecular weight filters, column purification, ultracentrifugation and/or electrodialysis. High affinity reagents include antibodies that selectively bind to high abundance polypeptides or reagents that have a specific pH, ionic value, or detergent strength. High molecular weight filters include membranes that separate molecules on the basis of size and molecular weight. Such filters may further employ reverse osmosis, nanofiltration, ultrafiltration and microfiltration.

Ultracentrifugation is an exemplary method for removing undesired polypeptides. Ultracentrifugation is the centrifugation of a sample at about 60,000 rpm while monitoring with an optical system the sedimentation (or lack thereof) of particles. Electrodialysis is another method for removing unwanted polypeptides. A manifold or microfluidics device can perform electrodialysis to remove high molecular weight polypeptides or undesired polypeptides. Electrodialysis can be used first to allow only molecules under approximately 30 kD to pass through into a second chamber. A second membrane with a very small molecular weight (roughly 500 D) allows smaller molecules to egress the second chamber.

Upon preparation of the samples, metabolic products of interest may be separated in another embodiment of the invention. Separation can take place in the same location as the preparation or in another location. In one embodiment of the invention, separation occurs in the same microfluidics device where preparation occurs, but in a different location on the device. Samples can be removed from an initial manifold location to a microfluidics device using various means, including an electric field. In another embodiment of the invention, the samples are concentrated during their migration to the microfluidics device using reverse phase beads and an organic solvent elution such as 50% methanol. This elutes the molecules into a channel or a well on a separation device of a microfluidics device.

Chromatography constitutes another method for separating subsets of substances. Chromatography is based on the differential absorption and elution of different substances. Liquid chromatography (LC), for example, involves the use of fluid carrier over a non-mobile phase. Conventional LC columns have an in inner diameter of roughly 4.6 mm and a flow rate of roughly 1 ml/min. Micro-LC has an inner diameter of roughly 1.0 mm and a flow rate of roughly 40 ul/min. Capillary LC utilizes a capillary with an inner diameter of roughly 300 im and a flow rate of approximately 5 ul/min. Nano-LC is available with an inner diameter of 50 um-1 mm and flow rates of 200 nl/min. The sensitivity of nano-LC as compared to HPLC is approximately 3700 fold. Other types of chromatography contemplated for additional embodiments of the invention include, without limitation, thin-layer chromatography (TLC), reverse-phase chromatography, high-performance liquid chromatography (HPLC), and gas chromatography (GC).

In another embodiment of the invention, the samples are separated using capillary electrophoresis separation. This method separates the molecules based on their eletrophoretic mobility at a given pH (or hydrophobicity).

In another embodiment of the invention, sample preparation and separation are combined using microfluidics technology. A microfluidic device is a device that can transport liquids including various reagents such as analytes and elutions between different locations using microchannel structures.

### Detection

In one embodiment of the invention, the sample may be delivered directly to the detection device without preparation and/or separation beforehand. In another embodiment of the invention, once prepared and/or separated, the metabolic products are delivered to a detection device, which detects them in a sample. In another embodiment of the invention, metabolic products in elutions or solutions are delivered to a detection device by electrospray ionization (ESI). In yet another embodiment of the invention, nanospray ionization (NSI) is used. Nanospray ionization is a miniaturized version of ESI and provides low detection limits using extremely limited volumes of sample fluid.

In another embodiment of the invention, separated metabolic products are directed down a channel that leads to an electrospray ionization emitter, which is built into a microfluidic device (an integrated ESI microfluidic device). Such integrated ESI microfluidic device may provide the detection device with samples at flow rates and complexity levels that are optimal for detection. Furthermore, a microfluidic device may be aligned with a detection device for optimal sample capture.

Detection devices can comprise of any device or experimental methodology that is able to detect metabolic product presence and/or level, including, without limitation, IR (infrared spectroscopy), NMR (nuclear magnetic resonance), including variations such as correlation spectroscopy (COSY), nuclear Overhauser effect spectroscopy (NOESY), and rotating frame nuclear Overhauser effect spectroscopy (ROESY), and Fourier Transform, 2-D PAGE technology, Western blot technology, tryptic mapping, in vitro biological assay, immunological analysis, LC-MS (liquid chromatography-mass spectrometry), and MS (mass spectrometry).

For analysis relying on the application of NMR spectroscopy, the spectroscopy may be practiced as one-, two-, or multidimensional NMR spectroscopy or by other NMR spectroscopic examining techniques, among others also coupled with chromatographic methods (for example, as LC-NMR). In addition to the determination of the metabolic product in question, ¹H-NMR spectroscopy offers the possibility of determining further metabolic products in the same investigative run. Combining the evaluation of a plurality of metabolic products in one investigative run can be employed for so-called “pattern recognition”. In one embodiment of the invention, the strength of diagnostic statements which the methods permit is improved by an evaluation in the pattern recognition mode as compared to the isolated determination of the concentration of one metabolic product.

For immunological analysis, for example, the use of immunological reagents (e.g. antibodies), generally in conjunction with other chemical and/or immunological reagents, induces reactions or provides reaction products which then permit detection and measurement of the whole group, a subgroup or a subspecies of the metabolic product(s) of interest. These immunological methods according to the invention may be carried out in practice along the lines of the method published by Smal and Baldo (Smal, M. A. et al. 1991, Lipids 26: 1130-1135; Baldo, B. A. et al. 1991, Lipids 26: 1136-1139). Reference is made to these publications.

In one embodiment of the invention, mass spectrometry is relied upon to detect metabolic products present in a given sample. In another embodiment of the invention, an ESI-MS detection device. Such an ESI-MS may utilizes a time-of-flight (TOF) mass spectrometry system. Quadrupole mass spectrometry, ion trap mass spectrometry, and Fourier transform ion cyclotron resonance (FTICR-MS) are likewise contemplated in additional embodiments of the invention.

In another embodiment of the invention, the detection device interfaces with a separation/preparation device or microfluidic device, which allows for quick assaying of many, if not all, of the metabolic products in a sample. A mass spectrometer may be utilized that will accept a continuous sample stream for analysis and provide high sensitivity throughout the detection process (e.g., an ESI-MS). In another embodiment of the invention, a mass spectrometer interfaces with one or more electrosprays, two or more electrosprays, three or more electrosprays or four or more electrosprays. Such electrosprays can originate from a single or multiple microfluidic devices.

In another embodiment of the invention, the detection system utilized allows for the capture and measurement of most or all of the metabolic products introduced into the detection device.

In another embodiment of the invention, the detection system allows for the detection of change in a defined combination (“composite”) of metabolic products.

### Signal Processing

In another embodiment of the invention, the output from a detection device can subsequently be processed, stored, and further analyzed or assayed using a bio-informatics system. A bio-informatics system may include one or more of the following, without limitation: a computer; a plurality of computers connected to a network; a signal processing tool(s); a pattern recognition tool(s); a tool(s) to control flow rate for sample preparation, separation, and detection.

The data processing utilizes mathematical foundations. In another embodiment of the invention, dynamic programming is used to align a separation axis with a standard separation profile. Intensities may be normalized, for example, by fitting roughly 90% of the intensity values into a standard spectrum. The data sets can then be fitted using wavelets designed for separation and mass spectrometer data. In yet another embodiment of the invention, data processing filters out some of the noise and reduces spectrum dimensionality, potentially allowing for pattern recognition.

Following data processing, pattern recognition tools can be utilized to identify subtle differences between phenotypic states. Pattern recognition tools are based on a combination of statistical and computer scientific approaches, which provide dimensionality reduction. Such tools are scalable.

### Methods of Treatment and Prevention

According to one embodiment of the invention, myocardial ischemia or myocardial infarction is treated in a subject. According to another embodiment of the invention, myocardial ischemia or myocardial infarction is prevented in a subject at risk for myocardial ischemia or myocardial infarction.

In one embodiment, myocardial ischemia or myocardial infraction is treated, as defined herein, by (i) administration of at least one metabolite whose level is found to be decreased in the subject, (ii) administration of at least one inhibitor of at least one metabolite whose level is found to be increased in the subject, or (iii) a combination of (i) and (ii).

A metabolite can be adaptive (protective of the host) or maladaptive (injurious to the host). Ostensibly, in one embodiment, treatment would comprise administering such an adaptive metabolite to the subject. However, a metabolite can be adaptive at a specific level (for example, malonic acid, glycerol-3-P, and inosine are protective of the host heart, vessel, or the like at certain levels, but become injurious to the host once present above a threshold level). Thus, in another embodiment, treatment could comprise administering an inhibitor to the same metabolite in a specific clinical scenario.

In another embodiment, myocardial ischemia or myocardial infraction is prevented in a subject at risk for myocardial ischemia or myocradial infarction by (i) administration of at least one metabolite whose level is found to be decreased in the subject, (ii) administration of at least one inhibitor of at least one metabolite whose level is found to be increased in the subject, or (iii) a combination of (i) and (ii).

Alternatively, the methods of treatment and/or prevention of the invention could comprise administering a compound that upregulates a pathway that results in the production of the metabolite in question.

The efficacy of disease treatment according to the invention may be assessed by monitoring changes in the disease state in subject receiving the at least one metabolite or the at least one inhibitor of at least one metabolite (or combination thereof) and comparing them to the progression or persistence of disease in control subjects who are treated with placebos (i.e. a pharmaceutically-acceptable carrier without the metabolite or inhibitor of the metabolite).

### Pharmaceutical Compositions Administration, Preparation, and Dosage

A metabolite and/or an agent that inhibits a metabolite can be administered to a subject by standard methods. For example, the metabolite and/or agent can be administered by any of a number of different routes including intravenous, intradermal, subcutaneous, oral (e.g., inhalation or ingestion), transdermal (topical), and transmucosal. In one embodiment, the agent is administered by injection, e.g., intra-arterially, intramuscularly, or intravenously.

The metabolite and/or metabolite inhibitor (also referred to herein as “active compound”) can be incorporated into pharmaceutical compositions suitable for administration to a subject, e.g., a human, typically including a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The carrier may further include an adjuvant. Adjuvants such as incomplete Freund's adjuvant, aluminum phosphate, aluminum hydroxide, or alum are materials well known in the art. The use of such media and agents for pharmaceutically active substances are known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and anti-fungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a metabolite and/or an agent that inhibits a metabolite) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In a preferred embodiment, the pharmaceutical composition is injected into an affected vessel, e.g., an artery, or an organ, e.g., the heart.

The pharmaceutical compositions of the present invention may be manufactured in a manner known in the art, e.g. by means of conventional mixing, dissolving, granulating, dragee-making, levitating, emulsifying, encapsulating, entrapping or lyophilizing processes.

The pharmaceutical composition may be provided as a salt and can be formed with many acids, including but not limited to hydrochloric, sulfuric, acetic, lactic, tartaric, malic, succinic, etc. . . . Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a Ph range of 4.5 to 5.5 that is combined with buffer prior to use.

After pharmaceutical compositions comprising a metabolite and/or agent that inhibits a metabolite formulated in a acceptable carrier have been prepared, they can be placed in an appropriate container and labeled for treatment of an indicated condition with information including amount, frequency, and method of administration.

The pharmaceutical compositions can be included in a container, pack, or dispenser together with instructions for administration. The dosages administered will vary from patient to patient. In the treatment (or prevention) of myocardial ischemia or myocardial infarction, a therapeutically effective dosage regimen should be used. By “therapeutically effective”, one refers to a treatment regimen sufficient to restore the subject to a state in which no myocardial ischemia or myocardial infarction is detectable. Alternatively, a “therapeutically effective” regimen may be sufficient to arrest or otherwise ameliorate symptoms of myocardial ischemia or myocardial infarction. Generally, in the treatment of myocardial ischemia or myocardial infarction, an effective dosage regimen involves providing the medication over a period of time to achieve noticeable therapeutic effects.

Generally, a therapeutic composition of the invention may be administered in a single dose in the range of from about 1 fg to about 1 g per kg body weight, preferably about 1 ug to about 1 mg per kg body weight. This dosage may be repeated daily, weekly, monthly, yearly, or as considered appropriate by the treating physician.

A therapeutic composition of use in the invention can be given in a single or multiple dose. A multiple dose schedule is one in which a primary course of administration can include 1-10 separate doses, followed by other doses given at subsequent time intervals required to maintain and or reinforce the level of the metabolite of inhibitor. Such intervals are dependent on the continued need of the recipient for the metabolite or inhibitor, and/or the half-life of the metabolite or inhibitor. The efficacy of administration may be assayed by monitoring the reduction in the levels of a symptom indicative or associated with myocardial ischemia or myocardial infarction. Alternatively, the efficacy of administration may be assayed by monitoring the level of the metabolite whose level had previously (previous to treatment) increased or decreased in the subject. The assays can be performed according to methods known to one skilled in the art.

### Kits

In another embodiment, the invention provides kits for monitoring and diagnosing myocardial ischemia or (early) myocardial infarction, wherein the kits can be used to detect the metabolic products described herein. For example, the kits can be used to detect any one or more of the metabolic products potentially differentially present in samples of the subjects before vs. after the administration of a controlled insult.

The kits of the invention may include instructions for the assay, reagents, testing equipment (test tubes, reaction vessels, needles, syringes, etc.), standards for calibrating the assay, and/or equipment provided or used to conduct the assay. The instructions provided in a kit according to the invention may be directed to suitable operational parameters in the form of a label or a separate insert.

In yet another embodiment, the invention provides kits for treating myocardial ischemia or (early) myocardial infarction. For example, the kits can be used to administer at least one metabolite or an inhibitor of at least one metabolite or a combination thereof to a subject.

This invention is further illustrated by the following examples, which should not be construed as limiting. A skilled artisan should readily understand that other similar instruments with equivalent function/specification, either commercially available or user modified, are suitable for practicing the instant invention. Rather, the invention should be construed to include any and all applications provided herein and all equivalent variations within the skill of the ordinary artisan.

### II. Examples

### Example 1

### Planned MI Recapitulates Spontaneous MI

Patients with Hypertrophic Obstructive Cardiomyopathy (HOCM) Undergoing Septal Ablation: Enrollment and Protocol

A total of 36 patients undergoing planned MI using alcohol septal ablation for the treatment of symptomatic hypertrophic obstructive cardiomyopathy (HOCM) were included. Inclusion criteria for the cohort were: 1) primary HOCM; 2) septal thickness of 16 mm or greater; 3) resting outflow tract gradient of greater than 30 mmHg, or an inducible outflow tract gradient of at least 50 mm Hg; 4) symptoms refractory to optimal medical therapy; and 5) appropriate coronary anatomy. The most proximal accessible septal branch was instrumented using standard angioplasty guiding catheters and guidewires and 1.5 or 2.0 mm×9 mm Maverick™ balloon catheters. Radiographic and echocardiographic contrast injections confirmed proper selection of the septal branch and balloon catheter position. Ethanol was infused through the balloon catheter at 1 ml per minute. Additional injections in the same or other septal branches were administered as needed, causing cessation of blood flow to the isolated myocardium, and to reduce the gradient to <20 mmHg.¹⁶

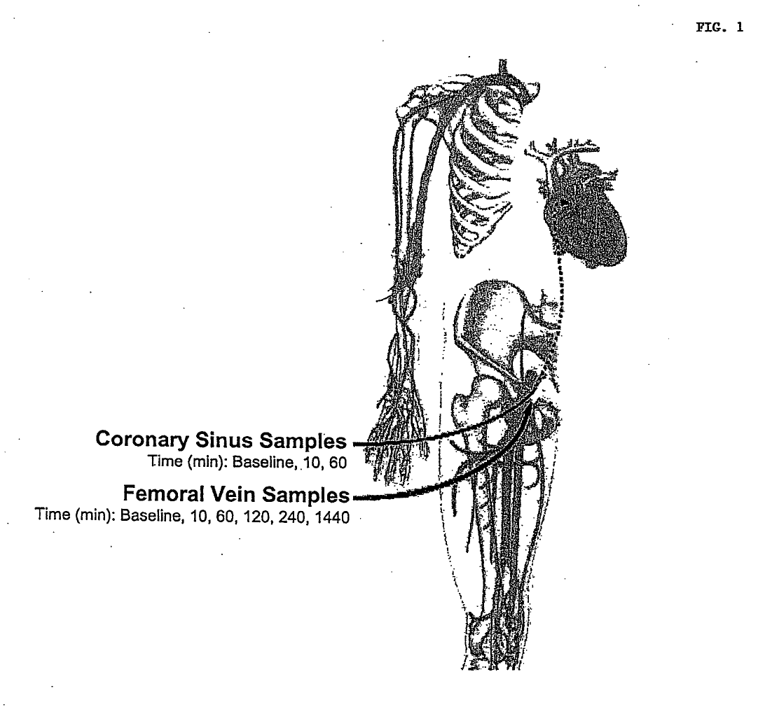

Blood was drawn at baseline (just prior to the onset of the ablation) and at minutes, 1 hour, 2 hours, 4 hours and 24 hours following the onset of injury as depicted in FIG. 1. 13 of the 36 patients consented to the placement of a coronary sinus catheter during the ablation, allowing for the simultaneous sampling from coronary sinus and femoral catheters at the baseline, 10-minute and 1-hour time points. The coronary sinus catheter was subsequently removed prior to the patient leaving the catheterization suite.

### Blood Sample Processing

Samples were obtained from femoral venous catheters during the procedure or, where indicated, from a catheter placed in the coronary sinus. Samples were collected in K₂EDTA-treated tubes (Becton Dickinson). All blood samples were centrifuged at 2000×g for 10 minutes to pellet cellular elements. The supernatant plasma was then aliquoted and immediately frozen at −80° C. to minimize freeze-thaw degradation. Additional blood samples were sent to the clinical chemistry laboratory for evaluation of the standard cardiac markers creatine kinase (CK), CK-MB, and Troponin T (Roche Diagnostics).

### High Performance Liquid Chromatography (HPLC) and Mass Spectrometry Analysis

For the analysis of sugars, ribonucleotides, and organic acids, 200 μl of plasma was subjected to ethanol precipitation (80% ethanol, 19.9% H₂O, 0.1% formic acid) at 4° C. for 2 hours, centrifuged at 15000×g for 15 minutes, and the supernatant evaporated in a nitrogen-chamber at 30° C. (Caliper Life Sciences). Samples were reconstituted in 60 μl H₂O, and aliquots were separated sequentially and automatically by injection onto three HPLC columns with orthogonal separation characteristics, as previously described.¹⁰ Sugars and ribonucleotides were separated on a Luna amino column (Phenomenex) under normal phase using acetonitrile/water/0.25% ammonium hydroxide/10 mM ammonium acetate at pH 11 in a run time of 3.5 minutes. Organic acids were separated using a Synergi Polar-RP column (Phenomenex) under reverse phase using acetonitrile/water/5 mM ammonium acetate at pH 5.6-6.0 in a run time of 3.5 minutes. For the analysis of amino acids, plasma was diluted 10-fold with H₂O and then subjected to reverse phase chromatography on a Luna phenyl-hexyl column (Phenomenex) using acetonitrile/water/0.1% acetic acid at pH 3.5-4.0 in a run time of 1.5 minutes.

The three HPLC columns were connected in parallel via an automated switching valve on a robotic sample loader (Leap Technologies) to a triple quadrupole mass spectrometer (AB4000Q, Applied Biosystem/Sciex), operated in electrospray ionization mode using a turbo ion spray LC/MS interface. Either positive or negative ions were selected for targeted MS/MS analysis using selective reaction monitoring (SRM) conditions. A total of 210 known metabolites were monitored for each sample. Precursor and product ions of metabolites presented in tables are detailed in Table 1, below. Data acquisition parameters are shown for multiple reaction monitoring experiments for all metabolites described in the text. All precursor and product ions are singly charged.

TABLE 1

| | | Precursor- | Product- | Collision |
|---|---|---|---|---|
| Metabolite | Polarity | ion mass | ion mass | Energy (eV) |
| Trimethylamine-N-Oxide | Positive | 76.1 | 58.0 | 29 |
| Lactic Acid | Negative | 89.0 | 43.0 | −20 |
| Glyceraldehyde | Negative | 89.0 | 59.0 | −10 |
| Alanine | Positive | 90.0 | 44.0 | 17 |
| Acetoacetate | Negative | 101.0 | 57.0 | −15 |
| Malonic Acid | Negative | 103.0 | 59.0 | −15 |
| Choline | Positive | 104.1 | 60.0 | 27 |
| Aminoisobutyric Acid | Positive | 104.1 | 86.0 | 16 |
| Serine | Positive | 106.0 | 60.0 | 18 |
| Proline | Positive | 116.1 | 70.0 | 20 |
| Succinic Acid | Negative | 117.0 | 73.0 | −20 |
| Threonine | Positive | 120.1 | 74.0 | 18 |
| Taurine | Positive | 126.0 | 108.0 | 18 |
| 1-methylhistamine | Positive | 126.1 | 109.0 | 22 |
| Isoleucine/Leu | Positive | 132.1 | 86.2 | 18 |
| Creatine | Positive | 132.1 | 90 | 17 |
| Malic Acid | Negative | 133.0 | 115.0 | −20 |
| Hypoxanthine | Negative | 135.0 | 92.0 | −23 |
| Mevalonic Acid/Lactone | Negative | 147.1 | 59.0 | −19 |
| Glutamine/Lysine | Positive | 147.1 | 84.0 | 25 |
| Glutamic Acid | Positive | 148.1 | 84 0 | 23 |
| Xanthine | Negative | 151.0 | 108.0 | −34 |
| 3-OH-Anthanilic Acid | Positive | 154.0 | 136.2 | 18 |
| Orotic Acid | Negative | 155.0 | 111.0 | −22 |
| Carnitine | Positive | 163.1 | 85.0 | 29 |
| Glycerol-3-P | Negative | 171.0 | 79.0 | −22 |
| Aconitic Acid | Negative | 173.0 | 129 | −8 |
| Glycerate-2-P | Negative | 185.0 | 79.1 | −20 |
| ADMA/SDMA | Positive | 203.1 | 70.3 | 40 |
| Ribose-5-P/Ribulose-5-P | Negative | 229.0 | 97.0 | −20 |
| Inosine | Negative | 267.1 | 135.0 | −30 |
| DCMP | Negative | 306.1 | 79.0 | −50 |
| AMP | Negative | 346.1 | 79.0 | −43 |

M/Z = mass/charge, P = phosphate, ADMA = asymmetric dimethylarginine, SDMA = symmetric dimethylarginine, DCMP = deoxycytidine monophosphate, AMP = adenosine monophosphate,

Metabolite quantification was performed by integrating peak areas for parent/daughter ion pairs using Multiquan software (Applied Biosystem/Sciex), and, subsequently, all metabolite peaks were manually reviewed for peak quality in a blinded manner prior to statistical analysis.

### Evaluation of Plasma Levels of Metabolites

Studies were undertaken using biochemical standards to establish the relationship between mass spectrometry-based intensity units and absolute changes in plasma metabolites. Specifically, it was evaluated whether the addition of exogenous metabolites to plasma samples would result in linear increases in mass-spectrometry intensity units in the range of changes observed in response to myocardial injury.

Six metabolites (two representatives from each of the 3 HPLC columns used for sample preparation) that demonstrated varying degrees of change in response to myocardial injury were chosen for further analysis (from Table 1, above). Because amino acids and amines were measured after simple plasma dilution, without drying down or reconstituting samples, it was possible to estimate absolute concentrations of these analytes from the y-intercept of the line determined in dose-response studies with exogenous standards.

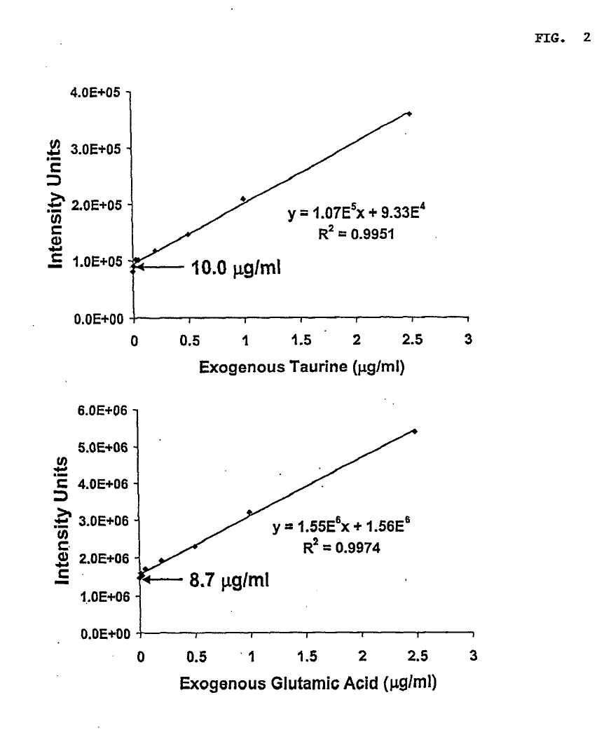

Reported concentrations of glutamic acid and taurine in normal human plasma range from 1.6-9.7 μg/ml and 5.0-13.5 μg/ml, respectively (Lepage, N., et al. 1997 Clin Chem 43:2397-402; Albert, J. D., et al. 1986 Am J Physiol 251: E604-10). In a pooled human plasma sample, glutamic acid (8.7 μg/ml) and taurine (10.0 μg/ml) concentrations (FIG. 2) were measured that were very similar to those previously reported. Furthermore, the dose-response relationship for each of the six compounds analyzed remained linear across a greater than 500% increase from reported plasma concentrations. Therefore, the observed increases in these compounds in response to injury, which ranged from 15% for taurine to 301% for hypoxanthine, correspond to similar increases in absolute concentrations of these metabolites.

In summary, standard addition experiments for representative metabolites taurine and glutamic acid added into human plasma allowed identification of the baseline circulating plasma levels of these metabolites and confirmed the linearity of the mass spectrometry quantitation (FIG. 2). Thus, studies using representative biochemical standards confirmed that the observed changes in metabolite peaks corresponded to linear increases in absolute concentrations of metabolites.

### Statistical Analysis

For clinical characteristics, values for continuous variables are presented as mean±SD, and comparisons between groups were performed using two-sample t-tests. Association between categorical variables was assessed using the Fisher's Exact Test.

A total of 36 patients with HOCM underwent planned MI with alcohol septal ablation. Clinical characteristics of the study population are detailed in Table 2, below.

TABLE 2
Baseline clinical characteristics of study subjects

| | Planned MI | Planned MI | | |
|---|---|---|---|---|
| | Derivation | Validation | Control Cath | Spontaneous |
| | Cohort | Cohort | Cohort | MI Cohort |
| | (n = 20) | (n = 16) | (n = 16) | (n = 12) |
| Age, years | 63 ± 14 | 60 ± 15 | 63 ± 13 | 64 ± 13 |
| Male sex, (%) | 40 | 50 | 63 | 75 |
| Caucasian Race, (%) | 90 | 94 | 83 | 78 |
| Creatinine baseline | 0.9 ± 0.2 | 1.0 ± 0.2 | 1.1 ± 0.3 | 1.1 ± 0.3 |
| Peak troponin T (ng/ml) | 5.0 ± 3.0 | 4.0 ± 2.0 | <0.01* | 8.8 ± 4.5* |
| Peak creatine kinase (U/L) | 1149 ± 509 | 1296 ± 1328 | 100 ± 25* | 2929 ± 383* |
| Peak creatine kinase-MB (ng/mL) | 187 ± 98 | 217 ± 102 | 3 ± 0.7* | 324 ± 138* |

Continuous variables are presented as mean ± SD, categorical variables are presented as percentage.
*indicates P < 0.05 as compared to each of the other 3 cohorts.

The mean age was 61±13 years and 56% of the patients were female. The septal ablation recapitulated important features of clinical MI, including typical chest pain and electrocardiographic changes, as well as the development of echocardiographic evidence of septal wall motion abnormalities, as previously described.¹³-15 The standard biochemical metrics of myocardial injury, CK-MB and troponin T, were within normal limits prior to septal ablation and increased to 200±98 ng/ml and 4.5±2.6 ng/ml, respectively. CK-MB peaked at 8.9±4.5 hours and cardiac troponin T at 14.9±8.0 hours after planned MI, time courses consistent with spontaneous MI.¹⁷

### Example 2

### Derivation and Validation of Early Metabolic Changes in Peripheral Plasma

Peripheral blood samples were studied across the range of time points available (10 minutes to 24 hours) to characterize metabolic alterations associated with planned MI in a derivation cohort of 20 patients. The left hand columns of Table 3A, below, describe the most significantly changed metabolites 10 minutes after the onset of myocardial injury.

TABLE 3A
Metabolite changes in the peripheral blood detected 10 minutes after myocardial injury

| | Derivation cohort | | | Validation cohort | | |
|---|---|---|---|---|---|---|
| Metabolite | Median Δ (IQR) | | P value | Median Δ (IQR) | | P value |
| Alanine | −18.2 | (−23.5, −13.6) | 0.0003 | −12.4 | (−18.5, −8.2) | 0.001 |
| Hypoxanthine | 189.6 | (88.9, 408.4) | 0.0005 | 249.6 | (105.1, 335.1) | 0.0006 |
| Isoleucine/Leucine | 7.0 | (3.6, 17.0) | 0.0016 | 7.4 | (0.8, 16.9) | 0.048 |
| MalonicAcid | 16.0 | (12.4, 93.0) | 0.0019 | 24.2 | (19.7, 57.4) | 0.001 |
| Aminoisobutyric Acid | 31.7 | (13.4, 46.9) | 0.0031 | 2.9 | (−20.8, 32.5) | 0.98 |
| Trimethylamine-N-Oxide | −15.5 | (−29.0, −8.9) | 0.0031 | −24.4 | (−33.7, −7.3) | 0.026 |
| Threonine | −10.0 | (−11.9, −5.2) | 0.0036 | −4.9 | (−11.3, 0.3) | 0.041 |

A total of 7 metabolites were observed that were significantly changed (nominal P<0.005) with an estimated false discovery rate of ˜13%. Based on this false discovery rate, it was expected that 6 of the 7 metabolites were truly differentially changed.

Because biomarker discovery studies are vulnerable to unintentional “overfitting” of data,¹⁸, 19 the performance of metabolites that were significantly changed in the derivation cohort were then assessed in an independent validation cohort of patients undergoing the septal ablation procedure (n=16, Table 3A, above, right columns). Significant changes in 6 of the 7 metabolites observed in the derivation cohort were noted in the validation cohort, as well (p<0.05). All of these metabolites showed concordance with the derivation cohort in the direction of change, and the magnitude of changes in the two cohorts were highly correlated (r²=0.87, P=0.01). Metabolic changes included products of purine and pyrimidine catabolism, hypoxanthine and malonic acid, respectively, as well as several amino acids. Of note, the alterations in these metabolites were seen when no significant rises in the clinically available biomarkers (CK-MB and troponin T) were detectable in the plasma (p=NS for both).

By 60 minutes after planned MI, more metabolic changes were noted (Table 3B, below).

TABLE 3B
Metabolite changes in the peripheral blood detected 60 minutes after myocardial injury

| | Derivation cohort | | | Validation cohort | | |
|---|---|---|---|---|---|---|
| Metabolite | Median Δ (IQR) | | P value | Median Δ (IQR) | | P value |
| Carnitine | 15.1 | (10.1, 24.1) | 0.0001 | 20.3 | (7.6, 45.0) | 0.012 |
| Alanine | −22.6 | (−24.8, −20.2) | 0.0002 | −11.9 | (−28.6, −7.1) | 0.012 |
| Hypoxanthine | 301.7 | (136.7, 536.1) | 0.0003 | 342.5 | (201.4, 390.7) | 0.0076 |
| Threonine | −7.5 | (−14.4, −3.1) | 0.0003 | −6.9 | (−13.3, −2.8) | 0.0037 |
| Trimethylamine-N-Oxide | −20.2 | (−27.2, −9.6) | 0.0007 | −32.4 | (−39.1, −17.7) | 0.028 |
| Inosine | 51.6 | (7.2-184.0) | 0.0011 | 35.7 | (−24.4, 145.0) | 0.110 |
| Aminoisobutyric Acid | 45.5 | (24.9, 65.3) | 0.0012 | 27.7 | (0.9, 43.0) | 0.071 |
| Glyceraldehyde | 14.4 | (4.1-38.2) | 0.0014 | 24.5 | (−6.1, 36.8) | 0.032 |
| Serine | −10.0 | (−18.8, −4.8) | 0.0015 | −8.9 | (−19.4, −0.6) | 0.034 |
| Isoleucine/Leucine | 10.6 | (2.1, 18.6) | 0.0017 | 13.3 | (3.2, 17.2) | 0.049 |
| Malonic Acid | 47.9 | (5.9, 96.2) | 0.0022 | 43.7 | (16.9, 86.0) | 0.003 |
| Choline | −9.4 | (−15.2, −1.1) | 0.0025 | −10.3 | (−14.2, 1.7) | 0.049 |
| Xanthine | 48.1 | (18.7, 82.9) | 0.0027 | 71.9 | (26.9, 119.3) | 0.004 |
| Proline | −4.6 | (−9.1, −2.8) | 0.0036 | −4.2 | (−15.4, 2.8) | 0.099 |
| 1-methylhistamine | 12.9 | (3.2, 23.0) | 0.004 | 33.2 | (−1.9, 67.0) | 0.028 |

Δ = % change from baseline; IQR = interquartile range
Isomers/metabolites of identical retention times and parent-daughter ion pairs e.g., Isoleucine/Leucine cannot be distinguished by the platform.

All of the metabolic changes documented at 10 minutes were also observed at 60 minutes, underscoring the consistency of the findings. A total of 15 metabolites observed were significantly changed (nominal P<0.005), with an estimated false discovery rate of 5%. Indeed, significant changes in 12 of the 15 metabolites observed in the derivation cohort were noted in the validation cohort, as well (p<0.05), with strong trends for the remaining three metabolites. The magnitude of changes in the two cohorts was highly correlated at this time point as well (r²=0.94, P<0.0001). By 60 minutes, additional changes in purine metabolites (xanthine and inosine), an inflammatory mediator (methylhistamine), as well as other amines and amino acids, were detected.

### Statistical Analysis

Preliminary studies were performed using sample preparation and mass spectrometry replicates of pooled human samples to assess the coefficient of variation (% CV; 100×Standard deviation/mean value of data set) for the metabolites in the platform. This analysis showed that the aggregate CV was ˜20%. From the 36 patients in whom peripheral samples were collected in the planned MI study, 20 patients were randomly selected for analysis as a derivation set. Levels of metabolites were tested for statistically significant percent change from baseline using the Wilcoxon signed-rank test. A significance threshold of P<0.005 was used in the derivation cohort, as this threshold would be expected to yield no more than 1 false positive discovery out of 210 metabolites analyzed, assuming independent hypotheses.

Metabolites that changed significantly at either the 10-minute or 1-hour time-points in the planned MI derivation cohort and did not change significantly (P>0.2 or the magnitude of the change in the control group <25% of that observed in the planned MI patients) between the same time points in the control cohort of patients undergoing diagnostic catheterization without MI were selected as candidate early biomarkers for testing in the planned MI validation cohort that consisted of 16 patients. Criteria for validation was P<0.05 by Wilcoxon signed-rank test with the direction of change concordant with that observed in the derivation cohort. The relationship between change in metabolites in the derivation and validation cohorts was assessed with a Spearman correlation coefficient. Data in all tables indicate median and interquartile ranges of percent change from baseline.

The specificity of the findings observed in the planned MI cohort was explored by examining blood samples from patients undergoing routine cardiac catheterization, without the induction of myocardial infarction that occurs in the unique ablation injury model.

Control Subjects and Patients with Spontaneous Myocardial Infarction: Enrollment and Protocol

A control cohort of 16 patients undergoing elective, diagnostic cardiac catheterization for cardiovascular disease, but not acute myocardial ischemia, was enrolled. Blood was drawn prior to the onset of cardiac catheterization and at 10 minutes and 1 hour after the procedure was begun. A cohort of 12 patients undergoing emergent cardiac catheterization for acute ST-segment elevation, spontaneous MI within 8 hours of symptom onset was also enrolled. For this cohort, blood samples were obtained in the coronary catheterization suite. Protocols for obtaining blood from patients in each of these cohorts were approved by the Massachusetts General Hospital IRB and all subjects gave written informed consent.

Whereas the metabolic changes in the derivation and validation cohorts were highly correlated, as noted above, there was no correlation between the derivation cohort and the catheterization control group overall (P=0.47 at 10 minutes; P=0.76 at 60 minutes). However, cardiac cauterization alone was associated with changes in three metabolites, tryptophan, tyrosine, and phenylalanine, at either the 10-minute or 60-minute time points (P<0.01). These three metabolites were, therefore, not included in Table 3 and were excluded from further analysis. Thus, metabolites with changes that are not specific to myocardial injury and that may, instead, reflect procedural events such as arteriotomy or catheter manipulation were eliminated using the appropriate patient controls.

### Example 3

### Kinetic Analysis of Metabolic Changes in Peripheral Plasma

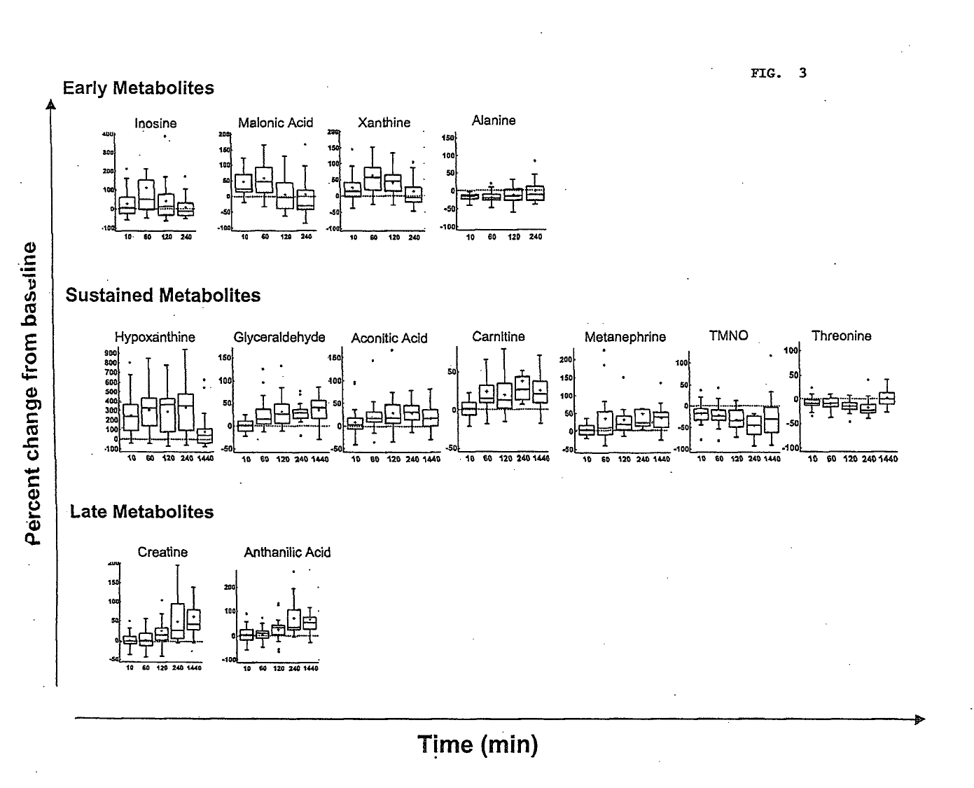

FIG. 3 demonstrates representative metabolites across a spectrum of time points after the planned MI. These kinetic data highlight early metabolic changes of potential clinical utility. Some metabolic changes were relatively transient (<120 minutes, e.g., alanine, inosine, xanthine, malonic acid; FIG. 3, upper panel), whereas other early metabolic changes persisted (≧240 minutes, e.g., hypoxanthine, glyceraldehyde, aconitic acid, trimethylamine-N-oxide, threonine, carnitine, and metanephrine; FIG. 3, middle panel). Later-appearing metabolites (FIG. 3, lower panel) included anthanilic acid and creatine, the latter of which was detected in a time course consistent with creatine kinase (CK-MB) release from cardiomyocytes undergoing necrosis.²⁰

### Example 4

### Metabolic Changes in Coronary Sinus Plasma

The specificity of the findings described above was further examined by exploring the anatomic origin of the metabolic changes. In a subgroup of 13 patients, metabolite levels were compared in samples obtained simultaneously from the peripheral blood and from a catheter placed in the coronary sinus, the venous outflow of the heart. This simultaneous sampling allowed the identification of transmyocardial enrichment or depletion of metabolites. Prior to myocardial injury, the coronary sinus and the peripheral blood metabolite levels were similar overall. In Tables 4A and 4B, below, the metabolites have been ranked by comparing the changes in the coronary sinus versus changes observed in the periphery at 10 and 60 minutes after injury.

TABLE 4A
Metabolites enriched in the coronary sinus 10 minutes after myocardial injury

| | Coronary Sinus | | P value | Peripheral | | P value | P value | Ratio |
|---|---|---|---|---|---|---|---|---|
| Metabolite | Median Δ (IQR) | | (vs no Δ) | Median Δ (IQR) | | (vs no Δ) | CS vs P | CS/P |
| Lactic Acid | 23.5 | (7.3, 40.7) | 0.021 | 0.06 | (−3.8, 8.4) | 0.97 | 0.010 | 394.5 |
| DCMP | 32.9 | (17.3, 77.4) | 0.0499 | −1.7 | (−28.1, 10.6) | 0.95 | 0.036 | 19.6 |
| AMP | 102.7 | (16.8, 161.4) | 0.0499 | 10.1 | (−7.5, 23.1) | 0.39 | 0.023 | 10.2 |
| Inosine | 86.9 | (9.3, 220.7) | 0.019 | 10.6 | (−18.7, 87.5) | 0.091 | 0.013 | 8.2 |
| Taurine | 15.1 | (11.8, 19.6) | 0.003 | −2.0 | (−6.3, 4.3) | 0.60 | 0.0084 | 7.3 |
| ADMA/SDMA | −14.9 | (−18.9, −7.8) | 0.0046 | −2.9 | (−20.2, 9.7) | 0.30 | 0.071 | 5.0 |
| Malic Acid | 29.9 | (19.5, 42.2) | 0.003 | 6.9 | (−1.2, 29.6) | 0.12 | 0.057 | 4.3 |
| Ribose-5-P/Ribulose-5-P | 18.1 | (2.70, 31.0) | 0.011 | −7.3 | (−11.5, 25.3) | 0.65 | 0.057 | 2.5 |
| Malonic Acid | 32.8 | (15.8, 112.8) | 0.0035 | 15.0 | (9.7, 69.2) | 0.026 | 0.17 | 2.2 |
| Hypoxanthine | 237.9 | (46.2, 313.7) | 0.011 | 128.1 | (6.8, 261.1) | 0.010 | 0.024 | 1.9 |
| Glutamine | 9.7 | (6.9, 12.6) | 0.003 | 5.8 | (−1.1, 9.3) | 0.26 | 0.011 | 1.7 |
| Glutamic Acid | 55.4 | (22.0, 68.5) | 0.002 | 37.6 | (18.8, 49.2) | 7.3E−05 | 0.060 | 1.5 |

TABLE 4B
Metabolites enriched in the coronary sinus 60 minutes after myocardial injury

| | Coronary Sinus | | P value | Peripheral | | P value | P value | Ratio |
|---|---|---|---|---|---|---|---|---|
| Metabolite | Median ± IQR Δ | | vs no Δ | Median ± IQR Δ | | (vs no Δ) | CS vs P | CS/P |
| Orotic Acid | 22.4 | (8.1-39.7) | 0.0063 | 0.87 | (−16.0, 16.5) | 0.36 | 0.01 | 25.9 |
| Succinic Acid | 14.3 | (−0.4, 25.7) | 0.027 | 2.1 | (−9.6, 11.6) | 0.47 | 0.043 | 6.9 |
| Glycerol-3-P | 31.3 | (1.2-65.4) | 0.0092 | −7.9 | (−17.8, 8.8) | 0.87 | 0.0063 | 4.0 |
| Glycerate-2-P | 12.7 | (1.7-50.6) | 0.016 | 3.1 | (−13.1, 13.4) | 0.85 | 0.042 | 4.0 |
| Taurine | 7.3 | (5.5, 13.1) | 0.001 | 2.1 | (−6.4, 4.3) | 0.60 | 0.041 | 3.4 |
| Malic Acid | 21.3 | (5.3, 35.3) | 0.0007 | 9.4 | (−0.2-17.1) | 0.10 | 0.002 | 2.3 |
| 1-methylhistamine | 17.0 | (9.7, 33.0) | 0.006 | 12.1 | (4.7, 30.7) | 0.05 | 0.19 | 1.4 |
| Isoleucine/Leucine | 13.8 | (0.5, 18.3) | 0.01 | 9.8 | (5.2, 17.6) | 0.003 | 0.078 | 1.4 |
| Hypoxanthine | 331.6 | (45.3, 560.7) | 0.0009 | 275.1 | (104.3, 407.2) | 0.0003 | 0.041 | 1.3 |

Δ = % change from baseline;
IQR = interquartile range
DCMP = deoxycytidine monophosphate,
AMP = adenosine monophosphate,
ADMA = asymmetric dimethylarginine,
SDMA = symmetric dimethylarginine,
P = phosphate
Isomers/metabolites of identical retention times and parent-daughter ion pairs e.g., ADMA/SDMA cannot be distinguished by the platform.

As a reference for comparison, B-type natriuretc peptide, a protein released from the heart in response to left ventricular wall stress, was enriched ˜1.3-fold in the coronary sinus samples 1 hour after injury (data not shown). Thus, metabolites with similar or higher enrichment are included in Tables 4A and 4B, above.

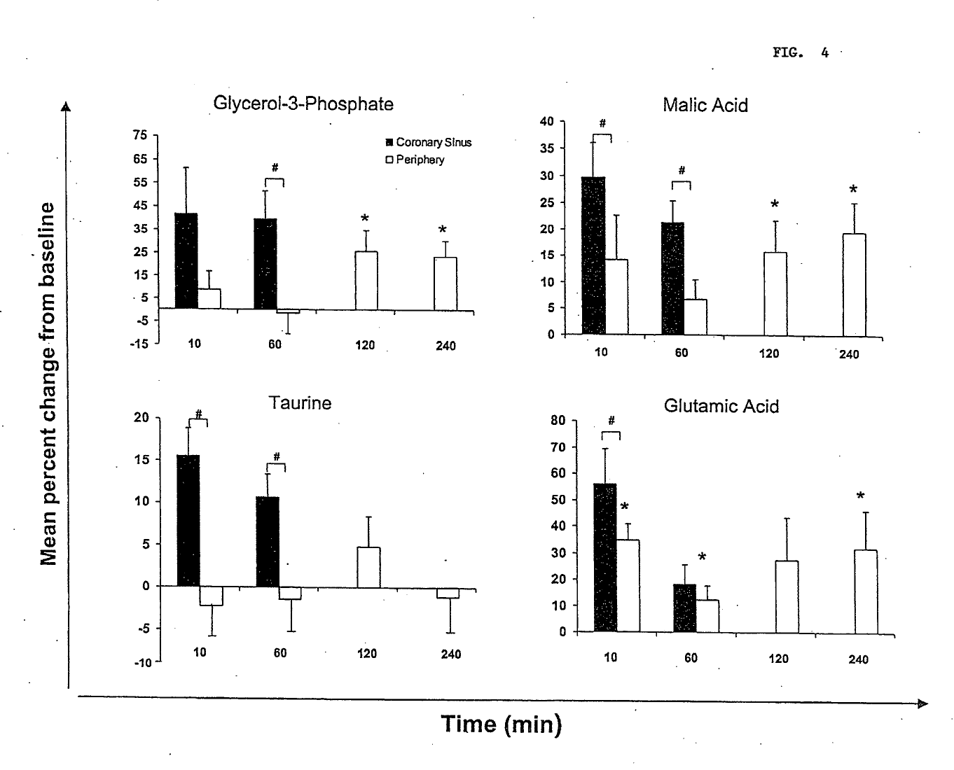

As expected, a transmyocardial enrichment pattern was evident for metabolites related to myocardial anaerobic metabolism, including lactic acid and succinic acid, as well as ATP degradation products such as hypoxanthine and AMP. Changes in the levels of certain metabolites were first apparent in the coronary sinus and were only later detected in the peripheral samples (FIG. 4 and Tables 4A and 4B, above). For example, levels of malic acid and glycerol-3-phosphate were significantly elevated by minutes in the coronary sinus, whereas, in the periphery, elevation was not evident until 120 minutes after injury (FIG. 4, top panel). In addition, cardiac-specific samples unmasked some metabolic changes that were not revealed in peripheral plasma [eg., taurine (FIG. 4, bottom left panel), succinic acid, asymmetrical/symmetrical dimethylarginine (ADMA/SDMA), orotic acid, and ribose-5-phosphate], perhaps due to rapid catabolism, wide distribution or excretion once circulated. Finally, other metabolites such as glutamic acid were concomitantly elevated in both the peripheral and coronary sinus blood samples at early time points, though to a greater degree in the coronary sinus (FIG. 4, bottom right panel).

### Statistical Analysis

Further analysis was carried out in the subgroup of 13 planned MI patients with matched coronary sinus and peripheral samples. Metabolites were considered to be “enriched” in the coronary sinus if they changed significantly in the coronary sinus at 10 or 60 minutes compared to baseline (P<0.05 using Wilcoxon signed-rank testing); and changed to a greater extent in the coronary sinus than in the periphery (median change in coronary sinus ≧1.3x median change in the periphery, P<0.05 by Wilcoxon signed-rank test).

To evaluate whether metabolic changes observed in the planned MI patients were generalizable to spontaneous MI, all of those metabolites that displayed significant changes from baseline at 1, 2 and 4 hours in the derivation and validation planned MI cohorts (P<0.05 at all three time points) were selected. A Wilcoxon Rank-Sum test was used to examine levels of these individual metabolites in the patients presenting with spontaneous MI, as compared to control patients presenting to the cardiac catheterization suite with non-acute cardiovascular disease. These metabolites were also compiled into a composite mass spectrometry intensity unit score for spontaneous MI and control patients. To ensure equal weighting of each metabolite in this composite score, the intensity values of each metabolite were rescaled to have a common median intensity of 1.0×10⁶ arbitrary units. The composite score was defined as the sum of metabolites that increased in planned MI minus the sum of metabolites that decreased in planned MI.

### Example 5

### Validation of Metabolic Markers in Spontaneous MI

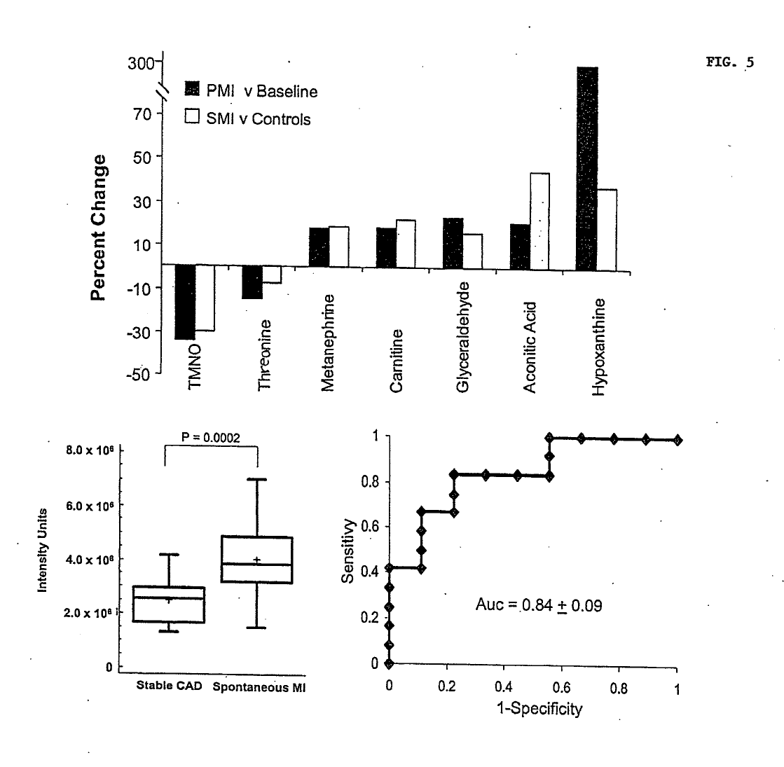

The applicability of the findings described herein to a cohort of patients with spontaneous MI presenting for acute coronary angiography was examined. Because the exact time of onset of spontaneous MIs relative to sample collection was heterogeneous (2.7±1.7 hours, range 0.5-8 hours), the group of metabolites that changed significantly in a sustained pattern after planned MI (FIG. 3, middle panel) were focused upon. These were the seven metabolites in the platform that were significantly changed at each of the 60, 120, and 240 minute time points in the planned MI cohort (P<0.05 for each time point; median changes across these three time points as compared to baseline are represented by the black bars in FIG. 5, left panel).

The difference in levels of each of these metabolites in the patients presenting with spontaneous MI were then examined, as compared to control patients presenting to the cardiac catheterization suite with non-acute cardiovascular disease (FIG. 5a, left panel, white bars). There was concordance of both the direction and magnitude of changes in the spontaneous MI cohort. A simple composite score was then generated by summing the equally weighted intensities for each of these metabolites to assess whether absolute mass spectrometry intensity units, in addition to relative changes, could distinguish spontaneous MI patients from controls (P=0.0002, FIG. 5b). This revealed excellent discriminatory ability with receiver-operating-characteristic (ROC) area under the curve (AUC) of 0.84 (FIG. 5c). The composite score further confirmed that metabolic biomarkers derived in the planned MI model were similarly altered in the spontaneous MI samples.

The results described herein demonstrate the novel application of a metabolomics platform to a carefully phenotyped patient cohort for the discovery of blood markers with the potential to detect the presence of very early myocardial injury. Abnormalities in circulating metabolites were identifiable as early as 10 minutes after myocardial injury, a time frame in which no currently used biomarkers are elevated. Beyond diagnostic utility, other metabolic signatures may be found to predict disease, to establish a reference for return to normality, and to aid in the design of new therapeutics for metabolic modulation.

### Example 6

### Metabolic Modulation of Cardiomyocyte Apoptosis

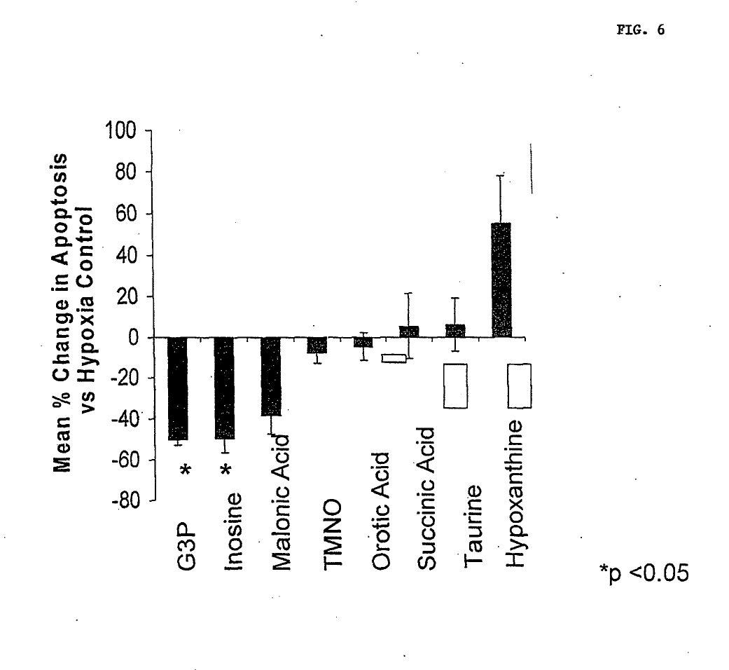

Neonatal rat cardiomyocytes were treated with individual metabolites previously identified using planned MI metabolomics. In the first, metabolites that are increased in the peripheral blood and/or the coronary sinus samples were prioritized. Neonatal cardiomyocytes were isolated from 1-day old rats, cultured for three days, and then challenged in a hypoxia chamber (<0.5% O2, 95% nitrogen and 5% CO₂ at 37° C.) for 3 hours, as previously described.¹⁸ Baseline apoptosis is typically ˜10%, which increases to ˜30% following hypoxic challenge, as assessed by FITC-conjugated annexin V staining. Metabolites at the indicated concentrations were added to experimental plates 5 minutes prior to the onset of hypoxia. Metabolites were examined at ˜2 fold reported blood concentrations (where available), as well as at one log increased concentration (see also Human Metabolomics Database, HMDB, at http://www.hmdb.ca). 30 minutes after reoxygenation, the number of annexin V-positive cells per five HPF was counted from n=3 culture plates. The data depicted represent the percent change in apoptosis relative to the hypoxia control for the lowest concentration of each metabolite screened. (*P<0.05 by unpaired student's t test). If the mean % change in apoptosis vs. hypoxia control is greater than zero (FIG. 6), the metabolite potentiates cell death. Accordingly, one would seek to inhibit the metabolite in question as part of treatment or prevention scheme. Such metabolites include, according to FIG. 6, succinic acid, taurine, and hypoxanthine.

If the mean % change in apoptosis vs. hypoxia control is less than zero (FIG. 6), the metabolite reduces cell death. Accordingly, one would seek to administer the metabolite in question as part of treatment or prevention scheme. Such metabolites include, according to FIG. 6, G3P, inosine, malonic acid, TMNO, and orotic acid. Alternatively, one could administer a compound that upregulates a pathway that results in the production of the metabolite in question.

### REFERENCES

1. Nicholson J K, Wilson I D. Opinion: understanding ‘global’ systems biology: metabonomics and the continuum of metabolism. Nat Rev Drug Discov 2003; 2:668-76.
2. Raamsdonk L M, Teusink B, Broadhurst D, et al. A functional genomics strategy that uses metabolome data to reveal the phenotype of silent mutations. Nat Biotechnol 2001; 19:45-50.
3. Allen J, Davey H M, Broadhurst D, et al. High-throughput classification of yeast mutants for functional genomics using metabolic footprinting. Nat Biotechnol 2003; 21:692-6.
4. An J, Muoio D M, Shiota M, et al. Hepatic expression of malonyl-CoA decarboxylase reverses muscle, liver and whole-animal insulin resistance. Nat Med 2004; 10:268-74.
5. Beecher C W W. The human metabolome. In: Harrigan G G, Goodacre R, eds. Metabolic profiling: its role in biomarker discovery and gene function analysis. Boston, Mass.: Kluwer Academic, 2003:pp. 311-319.
6. He W, Miao F J, Lin D C, et al. Citric acid cycle intermediates as ligands for orphan G-protein-coupled receptors. Nature 2004; 429:188-93.
7. Brindle J T, Antti H, Holmes E, et al. Rapid and noninvasive diagnosis of the presence and severity of coronary heart disease using 1H-NMR-based metabonomics. Nat Med 2002; 8:1439-44.
8. Kirschenlohr H L, Griffin J L, Clarke S C, et al. Proton NMR analysis of plasma is a weak predictor of coronary artery disease. Nat Med 2006; 12:705-10.
9. Lee M S, Kerns E H. LC/MS applications in drug development. Mass Spectrom Rev 1999; 18:187-279.
10. Sabatine M S, Liu E, Morrow D A, et al. Metabolic identification of novel biomarkers of myocardial ischemia. Circulation 2005; 112:3868-75.
11. Sigwart U. Non-surgical myocardial reduction for hypertrophic obstructive cardiomyopathy. Lancet 1995; 346:211-4.
12. Knight C, Kurbaan A S, Seggewiss H, et al. Nonsurgical septal reduction for hypertrophic obstructive cardiomyopathy: outcome in the first series of patients. Circulation 1997; 95:2075-81.
13. Lakkis N M, Nagueh S F, Kleiman N S, et al. Echocardiography-guided ethanol septal reduction for hypertrophic obstructive cardiomyopathy. Circulation 1998; 98:1750-5.
14. Lakkis N M, Nagueh S F, Dunn J K, Killip D, Spencer W H, 3rd. Nonsurgical septal reduction therapy for hypertrophic obstructive cardiomyopathy: one-year follow-up. J Am Coll Cardiol 2000; 36:852-5.
15. Yoerger D M, Picard M H, Palacios I F, Vlahakes G J, Lowry P A, Fifer M A. Time course of pressure gradient response after first alcohol septal ablation for obstructive hypertrophic cardiomyopathy. Am J Cardiol 2006; 97:1511-4.
16. Baggish A L, Smith R N, Palacios I, et al. Pathological effects of alcohol septal ablation for hypertrophic obstructive cardiomyopathy. Heart 2006; 92:1773-8.
17. Zimmerman J, Fromm R, Meyer D, et al. Diagnostic marker cooperative study for the diagnosis of myocardial infarction. Circulation 1999; 99:1671-7.
18. Ransohoff D F. Bias as a threat to the validity of cancer molecular-marker research. Nat Rev Cancer 2005; 5:142-9.
19. Ransohoff D F. Rules of evidence for cancer molecular-marker discovery and validation. Nat Rev Cancer 2004; 4:309-14.
20. Roberts R, Gowda K S, Ludbrook P A, Sobel B E. Specificity of elevated serum M B creatine phosphokinase activity in the diagnosis of acute myocardial infarction. Am J Cardiol 1975; 36:433-7.
21. Song D, O'Regan M H, Phillis J W. Mechanisms of amino acid release from the isolated anoxic/reperfused rat heart. Eur J Pharmacol 1998; 351:313-22.
22. Dorheim T A, Wang T, Mentzer R M, Jr., Van Wylen D G. Interstitial purine metabolites during regional myocardial ischemia. J Surg Res 1990; 48:491-7.
23. Delyani J A, Van Wylen D G. Endocardial and epicardial interstitial purines and lactate during graded ischemia. Am J Physiol 1994; 266:H1019-26.
24. Mei D A, Gross G J, Nithipatikom K. Simultaneous determination of adenosine, inosine, hypoxanthine, xanthine, and uric acid in microdialysis samples using microbore column high-performance liquid chromatography with a diode array detector. Anal Biochem 1996; 238:34-9.
25. Backstrom T, Goiny M, Lockowandt U, Liska J, Franco-Cereceda A. Cardiac outflow of amino acids and purines during myocardial ischemia and reperfusion. J Appl Physiol 2003; 94:1122-8.
26. Zemgulis V, Ronquist G, Bjerner T, et al. Energy-related metabolites during and after induced myocardial infarction with special emphasis on the reperfusion injury after extracorporeal circulation. Acta Physiol Scand 2001; 171:129-43.
27. Hassel B, Ilebekk A, Tonnessen T. Cardiac accumulation of citrate during brief myocardial ischaemia and reperfusion in the pig in vivo. Acta Physiol Scand 1998; 164:53-9.
28. Osterlund B, Andersson B, Haggmark S, et al. Myocardial ischemia induces coronary t-PA release in the pig. Acta Anaesthesiol Scand 2002; 46:271-8.
29. Mudge G H, Jr., Mills R M, Jr., Taegtmeyer H, Gorlin R, Lesch M. Alterations of myocardial amino acid metabolism in chronic ischemic heart disease. J Clin Invest 1976; 58:1185-92.
30. Srinivasan K N, Pugalendi K V, Sambandam G, Ramakrishna Rao M, Krishnan S, Menon V P. Comparison of glycoprotein components, tryptophan, lipid peroxidation and antioxidants in borderline and severe hypertension and myocardial infarction. Clin Chim Acta 1998; 275:197-203.
31. Wirleitner B, Rudzite V, Neurauter G, et al. Immune activation and degradation of tryptophan in coronary heart disease. Eur J Clin Invest 2003; 33:550-4.
32. Turgan N, Boydak B, Habif S, et al. Urinary hypoxanthine and xanthine levels in acute coronary syndromes. Int J Clin Lab Res 1999; 29:162-5.
33. Pisarenko O I, Baranov A V, Aleshin O I, et al. Features of myocardial metabolism of some amino acids and ammonia in patients with coronary artery disease. Eur Heart J 1989; 10:209-17.
34. Smolenski R T, de Jong J W, Janssen M, et al. Formation and breakdown of uridine in ischemic hearts of rats and humans. J Mol Cell Cardiol 1993; 25:67-74.
35. Kennergren C, Mantovani V, Lonnroth P, Nystrom B, Berglin E, Hamberger A. Extracellular amino acids as markers of myocardial ischemia during cardioplegic heart arrest. Cardiology 1999; 91:31-40.
36. Svedjeholm R, Ekroth R, Joachimsson P O, Ronquist G, Svensson S, Tyden H. Myocardial uptake of amino acids and other substrates in relation to myocardial oxygen consumption four hours after cardiac operations. J Thorac Cardiovasc Surg 1991; 101:688-94.
37. Keyzer J J, Breukelman H, Wolthers B G, Richardson F J, de Monchy J G. Measurement of N tau-methylhistamine concentrations in plasma and urine as a parameter for histamine release during anaphylactoid reactions. Agents Actions 1985; 16:76-9.
38. Jain M, Brenner D A, Cui L, et al. Glucose-6-phosphate dehydrogenase modulates cytosolic redox status and contractile phenotype in adult cardiomyocytes. Circ Res 2003; 93:e9-16.
39. Zimmer H G, Bunger R, Koschine H, Steinkopff G. Rapid stimulation on the hexose monophosphate shunt in the isolated perfused rat heart: possible involvement of oxidized glutathione. J Mol Cell Cardiol 1981; 13:531-5.
40. Zuurbier C J, Eerbeek 0, Goedhart P T, et al. Inhibition of the pentose phosphate pathway decreases ischemia-reperfusion-induced creatine kinase release in the heart. Cardiovasc Res 2004; 62:145-53.
41. Wallemacq P E, Vanbinst R, Asta S, Cooper D P. High-throughput liquid chromatography-tandem mass spectrometric analysis of sirolimus in whole blood. Clin Chem Lab Med 2003; 41:921-5.
42. Morrow D A, de Lemos J A, Sabatine M S, Antman E M. The search for a biomarker of cardiac ischemia. Clin Chem 2003; 49:537-9.

1. A method of detecting myocardial ischemia or myocardial infarction in a subject comprising detecting in a biological sample obtained from the subject a change in the amount of at least one member selected from the group consisting of malonic acid, asymmetric dimethyl arginine (ADMA), succinic acid, anthanilic acid, acetyl-CoA, asymmetric dimethyl arginine (ADMA)/symmetric dimethyl arginine (SDMA), and a metabolic product thereof, thereby detecting myocardial ischemia or early myocardial infarction in the subject.

2-8. (canceled)

9. The method of claim 1, wherein the change comprises an increase in the amount of at least one member of the group consisting of malonic acid, succinic acid, anthanilic acid, acetyl-CoA, asymmetric dimethyl arginine (ADMA)/symmetric dimethyl arginine (SDMA), and a metabolic product thereof.

10-15. (canceled)

16. The method of claim 1, wherein myocardial infarction is detected.

17. The method of claim 16, wherein the myocardial infarction is early myocardial infarction.

18. The method of claims 1, wherein myocardial ischemia is detected.

19. The method of claims 1, wherein the biological sample comprises a blood sample or a preparation thereof.

20. (canceled)

21. (canceled)

22. The method of claims 1, wherein the change is detected after administration of a controlled ischemic insult or planned myocardial infarction to the subject.

23. The method of claim 22, wherein the controlled ischemic insult comprises exercise testing, and the planned myocardial infarction comprises alcohol septal ablation for hypertrophic cardiomyopathy.

24-95. (canceled)

96. A method of detecting myocardial ischemia or myocardial infarction in a subject comprising detecting a decrease in the amount of at least one member of the group consisting of cholic acid, tyrosine, sucrose, trimethylamine-N-oxide, homoserine, threonine, choline, proline, 3-phosphoglyceric acid, and a metabolic product thereof in a biological sample obtained from the subject, thereby detecting myocardial ischemia or early myocardial infarction in the subject.

97. A method of detecting myocardial ischemia or myocardial infarction in a subject comprising detecting an increase in the amount of at least one member of the group consisting of Ile/Leu, taurine, aminoisobutyric acid, glyceraldehyde, xanthine, adenosine diphosphate (ADP), acetoacetate, carnitine, lactose, mevalonic acid lactone, 1-methylhistamine, glutamine, glutamic acid, orotic acid, glycerol-3-P, glycerate-2-P, adenosine monophosphate (AMP), malic acid, ascorbic acid, deoxycytidine monophosphate (DCMP), deoxycytidine diphosphate (DCDP), metanephrine, dimethyl glycine, creatine, ribose-5-P/ribulose-5-P, and a metabolic product thereof in a biological sample obtained from the subject, thereby detecting myocardial ischemia or early myocardial infarction in the subject.

98. (canceled)

99. The method of claim 96, wherein myocardial infarction is detected.

100. (canceled)

101. The method of claim 96, wherein myocardial ischemia is detected.

102. The method of claim 96, wherein the biological sample comprises a blood sample or a preparation thereof.

103. (canceled)

104. (canceled)

105. The method of claim 96, wherein the decrease or increase is detected after administration of a controlled ischemic insult or planned myocardial infarction to the subject.

106. The method of claim 105, wherein the controlled ischemic insult comprises exercise testing, and the planned myocardial infarction comprises alcohol septal ablation for hypertrophic cardiomyopathy.

107-119. (canceled)

120. The method of claim 97, wherein myocardial infarction is detected.

121. The method of claim 97, wherein myocardial ischemia is detected.

122. The method of claim 97, wherein the biological sample comprises a blood sample or a preparation thereof.

123. The method of claim 97, wherein the decrease or increase is detected after administration of a controlled ischemic insult or planned myocardial infarction to the subject.

124. The method of claim 123, wherein the controlled ischemic insult comprises exercise testing, and the planned myocardial infarction comprises alcohol septal ablation for hypertrophic cardiomyopathy.
